(19) United States
(12) Patent Application Publication
(10) Pub. No.: US 2009/0169593 A1
(43) Pub. Date: Jul. 2, 2009

(54) METHOD OF USING AND PRODUCING TROPOELASTIN AND TROPOELASTIN BIOMATERIALS

(75) Inventors: Kenton W. Gregory, Portland, OR (US); Robert Glanville, Beaverton, OR (US); Hooi-Sung Kim, Beaverton, OR (US); Rui-Qing Qian, Portland, OR (US); Carl Wamser, Portland, OR (US)

Correspondence Address: MARGER JOHNSON & MCCOLLOM, P.C., 210 SW MORRISON STREET, SUITE 400, PORTLAND, OR, 97204, US

(73) Assignees: BIOMEDICAL RESEARCH SERVICES, INC., Portland, OR (US); PROVIDENCE HEALTH SYSTEMS - Oregon, an Oregon Nonprofit Corporation, Seattle, WA (US)

(21) Appl. No.: 12/090,843

(22) PCT Filed: Oct. 19, 2006
(86) PCT No.: PCT/US06/60084

§ 371 Date: Dec. 18, 2008

Related U.S. Application Data
(60) Provisional application No. 60/728,471, filed on Oct. 19, 2005.

(51) Int. Cl. A61F 2/04; B05D 3/10

(52) U.S. Cl. 424/423; 427/2.24; 604/264; 623/1.46

(57) ABSTRACT

A device implantable within a human body, and a method for producing the device, are provided. The device comprises a biocompatible coating on at least a portion of an outer surface of a substrate. The biocompatible coating comprises tropoelastin. A biocompatible coating is formed in situ on the outer surface of the substrate.

### RELATED APPLICATION

This application is a non-provisional application of provisional application Ser. No. 60/728,471 filed Oct. 19, 2005. Priority of application 60/728,471 is hereby claimed. The entire contents of application 60/728,471 are hereby incorporated by reference.

### BACKGROUND OF THE INVENTION

This invention relates to methods for using tropoelastin, and to a method for producing tropoelastin biomaterials.

Elastic fibers are responsible for the elastic properties of several tissues such as skin and lung, as well as arteries, and are composed of two morphologically distinct components, elastin and microfibrils. Microfibrils make up the quantitatively smaller component of the fibers and play an important role in elastic fiber structure and assembly.

The most abundant component of elastic fibers is elastin. The entropy of relaxation of elastin is responsible for the rubber-like elasticity of elastic fibers. In vertebrates elastin is formed through the secretion and crosslinking of tropoelastin, the 72-kDa biosynthetic naturally occurring precursor to elastin. This is discussed, for example, in an article entitled “Oxidation, Cross-linking, and Insolubilization of Recombinant Crosslinked Tropoelastin by Purified Lysyl Oxidase” by Bedell-Hogan, et al in the Journal of Biological Chemistry, Vol. 268, No. 14, on pages 10345-10350 (1993).

Thirty to forty percent of atherosclerotic stenoses that are opened with balloon angioplasty restenose as a result of ingrowth of medial cells. Smooth muscle ingrowth into the intima appears to be more prevalent in sections of the artery where the internal elastic lamina (IEL) of the artery is ripped, torn, or missing, as in severe dilatation injury from balloon angioplasty, vessel anastomoses, or other vessel trauma that results in tearing or removal of the elastic lamina.

Prosthetic devices, such as vascular stents, have been used with some success to overcome the problems of restenosis or re-narrowing of the vessel wall resulting from ingrowth of muscle cells following injury. However, metal stents or scaffolds being deployed presently in non-surgical catheter based systems to scaffold damaged arteries are inherently thrombogenic and their deployment can result in catastrophic thrombotic closure. Metal stents have also been well demonstrated to induce a significant intimal hyperplastic response within weeks which can result in restenosis or closure of the lumen. Optimal arterial reconstruction would restore the arterial architecture such that normal vascular physiology and biology would be re-established thus minimizing acute and long-term maladaptive mechanisms of vascular homeostasis.

Damage to the arterial wall through disease or injury can involve the endothelium, internal elastic lamina, medial smooth muscle and adventitia. In most cases, the endogenous host response can repair and replace the endothelium, the smooth muscle and the adventitial layers over a period of weeks to months depending upon the severity of the damage. However, elastin does not undergo extensive post-developmental remodelling and the capacity for elastin synthesis declines with age. (see “Regulation of Elastin Synthesis in Organ and Cell Culture” by Jeffrey M. Davidson and Gregory C. Sephel in Methods in Enzymology 144 (1987) 214-232. Therefore, once damaged, elastic fibers are not substantially reformed. Neosynthesis of elastin in arterial walls subject to hypertension or neointimal hyperplasia represents the most significant example of post developmental elastin synthesis. This synthesis results in elastic structures mostly composed of elastin fibrils whose organization is unlike normal elastin architecture and probably contributes little to the restoration of normal vascular physiology.

In animal models of intimal hyperplasia or atherosclerosis it is well accepted that disruption of the internal elastic lamina is a prerequisite to reliable production of intimal hyperplasia or atherogenesis in large animals or primates. see Schwartz R. S., et al, in an article entitled “Restenosis After Balloon Angioplasty: Practical Proliferation Model In Porcine Coronary Arteries” in Circulation 1990:82:2190-2200. This observation is supported by several lines of evidence that suggest a role for elastin in the biological regulation of several cell types. Pathological studies indicate that elastin provides a secure attachment for endothelial cells and can act as a barrier to macromolecules such as mitogens and growth factors preventing these molecules from entering the media of blood vessels. Lipids, foamy macrophages, and other inflammatory cells do not appear to enter the intima as readily when a substantial and continuous elastin membrane is present immediately to the endothelium according to Sims, F. H., et al, in an article entitled “The Importance of A Substantial Elastic Lamina Subjacent To The Endothelium In Limiting the Progression of Atherosclerotic Changes” in Histopathology (1993) at 23:307-317. In addition, it has been shown by Ooyama, Toshiro and Sakamoto that chemotactic effects of soluble elastin peptides and platelet derived growth factor are inhibited by substratum bound elastin peptides. see “Elastase in the Prevention of Arterial Aging and the Treatment of Atherosclerosis. see “The Molecular Biology and Pathology of Elastic Tissues” edited by Chadwick, Derek J. and Jamie A. Goode, John Wiley and Sons Ltd, Chichester, England (1995). In vitro experiments show that alpha elastin suppresses the phenotypic transition (contractile to synthetic) of rabbit arterial SMC by interacting with a 130 kDa cell surface elastin binding protein for cell binding sequence VGVAPG. Rabbit smooth muscle cells adhering to elastic fibers appears to favor the contractile over the synthetic state which is identified with restonotic responses to injury. see “Changes in Elastin Binding Proteins During Phenotypic Transition of Rabbit Arterial Smooth Muscle Cells in Primary Culture” by Yamamoto, et al in Experimental Cell Research 218 (1995) pg. 339-345. Similar work by Ooyama and colleagues has demonstrated that the phenotypic change of smooth muscle cells from the contractile to the modified type is significantly retarded when the cells are grown on elastin coated dishes.

The invention makes possible tissue prostheses (particularly, vascular prostheses) that are essentially free of problems associated with prostheses known in the art.

Arterial replacement or reconstruction using tropoelastin based biomaterials not only may provide normal strength and elasticity but also may encourage normal endothelial re-growth, inhibit smooth muscle cell migration and thus restore normal vascular homeostasis to a degree not currently possible with synthetic grafts.

Metal stents or scaffolds are also being deployed presently in non-surgical catheter based systems to damaged arteries, however metal is inherently thrombogenic and can induce a significant intimal hyperplastic response. Optimal arterial reconstruction would restore the arterial architecture such that normal vascular physiology would be re-established thus minimizing acute and long-term maladaptive mechanisms of vascular homeostasis. Damage to the arterial wall through disease or injury can involve the endothelium, internal elastic lamina, medial smooth muscle and adventitia. In most cases, the endogenous host response can repair and replace the endothelium, the smooth muscle and the adventitial layers over a period of weeks to months depending upon the severity of the damage. The internal elastic lamina however, once disrupted or damaged, is not reconstituted. In addition to an important structural role inelasticity and strength of the vessel wall, the elastic lamina has also been thought to act as an inhibitor to smooth muscle cell in-growth and also as a barrier to macromolecules, such as mitogens and growth factors in the blood stream. In animal models of intimal hyperplasia or atherosclerosis, it is well accepted that disruption of the internal elastic lamina is a prerequisite to reliable production of intimal hyperplasia or atherogenesis in large animals or primates.

Tissue substitutes based upon elastin, a natural extracellular matrix protein that provides tissue elasticity and strength have been developed and tested in chronic long-term animal models for vascular, urethral, duodenal, esophageal and tympanic membrane repair. Antibiotics, coagulants, analgesics or other drugs have been incorporated to allow medical treatment with controlled release at the implantation site, having high local concentrations and low systemic concentrations.

### SUMMARY OF THE INVENTION

Devices implantable within a human body, and methods for producing the devices, are provided. In various embodiments of the present invention, a device comprises a biocompatible coating on at least a portion of an outer surface of a substrate, wherein the biocompatible coating comprises tropoelastin. In one embodiment the biocompatible coating is formed in situ on the outer surface of the substrate. In another embodiment, the biocompatible coating which is formed on at least a portion of an outer surface of the substrate comprises a polymer consisting essentially of tropoelastin.

In a further embodiment, a biocompatible coating which is formed in situ on at least a portion of an outer surface of the substrate by cross-linking tropoelastin on the outer surface of the substrate. In still a further embodiment cross-linking tropoelastin on the outer surface of the substrate is accomplished by introducing the substrate into a cross-linking solution. In an embodiment of this invention, the substrate is introduced by dipping same into a cross-linking solution.

In various embodiments, a biocompatible coating formed on at least a portion of an outer surface of the substrate comprises cross-linking tropoelastin monomers to form a polymer consisting essentially of tropoelastin. Exemplary agents for cross-linking tropoelastin include bi-functional with amino reactive functional groups. In various embodiments, the cross-linker may be a member the family of N-Hydroxysuccinimide-esters. For example, the cross-linker may be a selected one of Bis(sulfosuccinimidyl)glutarate, Bis(sulfosuccinimidyl)suberate, Disuccinimidyl glutarate, Disuccinimidyl suberate. In other embodiments, the cross-linker may be a selected one of 1-Ethyl-3-(3-dimethylaminopropyl) carbodiimide hydrochloride and glutaraldehyde.

In various embodiments, a biocompatible coating formed on at least a portion of an outer surface of the substrate comprises applying tropoelastin monomers on the outer surface of the substrate using techniques such as dip coating, spraying, or electrospinning.

The cross-linking solution can preferably further comprise a solvent capable of substantially preventing redissolution of the tropoelastin. In an embodiment herein a water immiscible solvent is employed. Preferred solvent materials for substantially preventing redissolution of the tropoelastin include immiscible solvent with aqueous solvent. In various embodiments, the solvent may be an organic solvent. Exemplary solvents include hydrocarbon solvents, ethers, chloroform, dichloromethane, and ethyl acetate.

In various embodiments, the cross-linking solution may also comprise a cross-linking agent. Exemplary agents for cross-linking tropoelastin include bi-functional with amino reactive functional groups. In various embodiments, the cross-linker may be a member the family of N-Hydroxysuccinimide-esters. For example, the cross-linker may be a selected one of Bis(sulfosuccinimidyl)glutarate, Bis(sulfosuccinimidyl)suberate, Disuccinimidyl glutarate, Disuccinimidyl suberate. In other embodiments, the cross-linker may be a selected one of 1-Ethyl-3-(3-dimethylaminopropyl) carbodiimide hydrochloride and glutaraldehyde.

In an embodiment of the present invention, a biocompatible coating which is formed in situ on at least a portion of an outer surface of the substrate comprises an intermediate bonding layer on at least a portion the outer surface of the substrate. The tropoelastin is adhered to an outer surface of the intermediate bonding layer. In another embodiment, adhering tropoelastin to an outer surface of the intermediate bonding layer comprises covalently bonding tropoelastin to the outer surface of the intermediate bonding layer.

The intermediate bonding layer, in various embodiments, comprises amine groups for cross-linking tropoelastin to the outer surface of said substrate. In such embodiments, the intermediate bonding layer may comprise an aminosilane for cross-linking tropoelastin to the outer surface of said substrate. The aminosilanes for cross-linking tropoelastin can include 3-(N-styrylmethyl-2-aminoethylamino)propyltrimethoxylsilane, N-phenylaminopropyltrimethoxylsilane, N-phenylaminomethylethoxylsilane, N-methylaminopropyltrimethoxylsilane, N-methylaminopropylmethyldimethoxylsilane, N-(3-methacryloxy-2-hydroxypropyl)-3-aminopropyltriethoxylsilane, N-(hydroxyethyl)-N-methylaminopropyltrimethoxylsilane, N-ethylaminoisobutylrimethoxylsilane, N-ethylaminoisobutylrimmethyldiethoxylsilane, 3-(2,4-dinitrophenylamino)propyltriethoxylsilane, 3-(1,3-dimethylbutylidene)aminopropyltriethoxylsilane, (N,N-dimethylaminopropy)trimethoxylsilane, dimethylaminomethylethoxylsilane, (N,N-diethyl-3-aminopropyl)trimethoxylsilane, diethylaminomethylriethoxylsilanel, N-cyclohexylaminopropyltrimethoxylsilane, t-butylaminopropyltrimethoxylsilane, bis(2-hydroxyethyl)-3-aminopropyltriethoxylsilane, 1,3-bis(3-aminopropyl)tetramethyldisiloxane, 1,3-bis(2-aminoethylaminomethyl) tetramethyldisiloxane, 11-aminoundecyltriethoxysilane, 3-aminopropyltris(trimethylsiloxy)silane, 3-aminopropyltris(methoxyethoxyethoxy)silane, 3-aminopropyltrimethoxylsilane, 3-aminopropyltriethoxylsilane, 3-aminopropylsilanetriol, 3-aminopropylpentamethyldisiloxane, 3-aminopropylmethyldiethoxysilane, 3-aminopropyldimethylethoxysilane, 3-aminopropylmethylbis(trimethylsiloxy)silane, 3-aminopropyldiisopropylethoxysilane, N-3-[amino(polypropylenoxy)]aminopropyltrimethoxysilane, o-aminophenyltrimethoxysilane, p-aminophenyltrimethoxysilane, m-aminophenyltrimethoxysilane, 3-(m-aminophenoxy)propyltrimethoxysilane, N-(2-aminoethyl)-11-aminoundecyltrimethoxysilane, N-(6-aminohexyl)aminopropyltrimethoxysilane, and N-(6-aminohexyl)aminomethyltriethoxysilane, etc.

In various embodiments, the substrate is may be pretreated prior to forming the biocompatible coating to form a pretreated substrate which facilitates adhering of the biocompatible coating thereto. In various embodiment,s pretreating the substrate prior to forming the biocompatible coating comprises oxidizing the substrate. Exemplary methods of oxidizing the substrate include electrochemical oxidation in acids and chemical oxidation or etching.

In another embodiment, oxidizing the substrate comprises electrochemical oxidation. Example of preferred electrochemical oxidation techniques include electrochemical oxiation in acids with negative and positive polarizing voltage.

In one embodiment, the substrate is formed of a metallic material. The substrate can also be formed of a non-metallic material, in an embodiment such as a polymer material or the like. In another embodiment, the substrate is a prosthetic device. In a further embodiment, the substrate is a stent, a conduit or a scaffold. For example, a conventional metallic prosthetic device, such as a stainless steel stent, has a contact angle of about 60 degrees. A description of the term “contact angle” will be hereinafter be provided. In general, a contact angle is the angle at which a liquid interface meets the solid surface and is typically measured using drops of distilled water at pH 7.0. In an embodiment of this invention, the substrate is pretreated to substantially reduce its hydrophilicity. The contact angle of a substrate is a measure of its hydrophilicity. On extremely hydrophilic surfaces, a water droplet may completely spread (an effective contact angle of 0°). On highly hydrophobic surfaces, which are incompatible with water, one may observe a large contact angle (70° to 90°). Some surfaces have water contact angles as high as 150° or even 180°.

Preferably, the pretreated substrate has a contact angle which is not more than about 50%, more preferably not more than about 40%, and most preferably not more than about 30%, of the contact angle of the unpretreated substrate prior to pretreatment. With respect to a substrate coated with a biocompatible coating, the contact angle is increased to increase hydrophilicity. Therefore, in one embodiment a substrate coated with a biocompatible coating has a contact angle which is at least about 150%, in another embodiment is at least about 175%, and in a further embodiment is at least about 200%, of the contact angle of the untreated substrate prior to pretreatment.

Preferably, the tropoelastin is arranged to form poly-tropoelastin aggregates prior to forming the biocompatible coating in situ on at least a portion of an outer surface of the substrate. In one embodiment, this is accomplished by coacervating the tropoelastin prior to forming of the biocompatible coating. Other preferred arrangement techniques may include electrospinning,

The biocompatible coating can be formed in nonuniform multiple layers on the surface of the substrate. However, in an embodiment of this invention, the biocompatible coating is formed in a substantially single biocompatible layer onto the substrate.

A drug can be incorporated into the biocompatible coating thereby decreasing the need for systemic intravenous or oral medications. Preferably, the biocompatible coating includes a drug for use in the human body.

### BRIEF DESCRIPTION OF THE DRAWINGS

FIGS. 1(a)-(d) depict the contact angles of water on a substrate, more particularly, a flat stainless steel surface.

FIG. 2 are cross-sectional SEM images of tropoelastin-coated stent: tropoelastin film side (spectrum 1); stainless steel side (spectrum 2); interface area between metal and tropoelastin film (spectrum 3).

FIG. 3 are EDX spectra of tropoelastin film side (spectrum 1); stainless steel side (spectrum 2); interface area between metal and tropoelastin film (spectrum 3).

FIGS. 4(a)-(c) are C1s XPS spectra.

FIG. 5 depicts atomic force microscope (AFM) images.

FIG. 6. shows atomic force microscope (AFM) images of (a) an uncoated stent and (b) a centrifugally treated dip-coated stent.

FIG. 7 are atomic force microscope (AFM) images of the inside of a centrifugally treated dip-coated stent (a) and the outside surface.

FIG. 8 shows scanning electron microscope (SEM) images of a dip-coated and crosslinked stent.

FIG. 9 shows an SEM image of a dip-coated and crosslinked stent that was treated centrifugally.

FIG. 10 is SEM images of a centrifugally treated dip-coated stent before and after expansion under water.

FIG. 11 are SEM images of the surface of an expanded and γ-irradiated coated stent.

FIG. 12(a)-(f) are SEM images of (a) an uncoated stent, (b) a crosslinked tropoelastin-coated stent before implantation, and (c-f) coated stents after two hours implantation.

### DETAILED DESCRIPTION

### Monomer Synthesis

Tropoelastin monomer is the soluble biosynthetic which is the naturally occurring precursor to elastin. It is formed naturally in vetebrates. Tropoelastin can be isolated from the aortas of copper deficient swine by known methods such as described by E. B. Smith, Atherosclerosis 37 (1980) tropoelastin is a 72-kDa polypeptide which is rich in glycine, proline, and hydrophobic amino acids. The exact amino acid composition of tropoelastin differs from species to species. Any polypeptide moiety that has art-recognized homology to tropoelastin can be considered a tropoelastin monomer for the invention.

The tropoelastin can be isolated from mammalian tissue or produced using recombinant expression systems. Furthermore, tropoelastin splice variants from any species can also be used for the invention.

The following are exemplary descriptions of methods of producing tropoelastin monomers used in the invention:

1. Tropoelastin can be extracted from mammals which have been placed on copper deficient or lathyritic diets. The deficiency of copper in the mammalian diet inhibits lysyl oxidase resulting in the accumulation of tropoelastin in elastin rich tissues. Copper deficient animals are grown rapidly on a diet composed largely of milk products and must be kept isolated from contaminating sources of copper. The protocol for raising copper deficient swine is detailed by L. B. Sandberg and T. B. Wolt. Production of Soluble Elastin from Copper Deficient Swine. Methods in Enzymology 82 (1982) 657-665. 150 mg of tropoelastin can be extracted from a 15-kg copper-deficient swine.

2. In a method similar to copper deficiency method in No. 1 above, feeding animals chemicals that effectively inhibit the action of lysyl oxidase (lathyrogens) also restricts the conversion of tropoelastin to amorphous elastin. This method produces similar yields of tropoelastin to copper-deficient swine. However, the special cages, water and diet required to raise copper-deficient animals are not required herein. To induce lathyrisim, animal diets are supplemented with 0.1% by weight α-aminoacetonitrile-HCl and 0.05% α-aminocaproic acid as described by Celeste B. Rich and Judith Ann Foster, Isolation of Soluble Elastin-Lathyrism. Methods in Enzymology 82 (1982) 665-673.

3. Tropoelastin can also be produced by mammalian cell culture systems. Short term cultivation of bovine vascular endothelial cells, nuchal ligament fibroblasts from cows and sheep, human skin fibro-blasts, and vascular smooth muscle cells from pigs and rabbits results in the accumulation of tropoelastin in the culture medium.

4. Recombinant tropoelastin produced by aprotein expression system is the preferred monomer for the invention. Recombinant protein technology is the transfer of recombinant genes into host organisms that grow and convert nutrients and metabolites into recombinant protein products. Using this technology, cDNA encoding tropoelastin can be cloned and expressed in protein expression systems to produce biologically active recombinant tropoelastin. Functionally distinct hydrophobic domains and lysine rich crosslinking domains are encoded in separate exons. This existence of multiple splice variants of tropoelastin in several species can be attributed to Cassette-like alternative splicing of elastin pre-mRNA. Expression of different recombinant splice variants of tropoelastin can produce proteins with distinct qualities. In addition, site directed in vitro mutagenesis can be used to alter the polypeptide sequence of the naturally occurring gene, thus creating alternate polypeptides with improved biological activity and physical properties. Expression of the full length elastin cDNA clone, cHEL2 and subsequent purification of recombinant human tropoelastin (rTE) has been achieved by Joel Rosenbloom, William R. Abrams, and Robert Mecham. Extracellular Matrix 4: The Elastic Fiber. The Faseb Journal 7 (1993) 1208-1218. rTE produced by the methods of Rosenbloom et. al. can be used for the invention, however, the methods are not considered to be part of the present invention. In addition, the invention is not limited to rTE produced from the expression of cHEL2. rTE produced from the expression of any tropoelastin genomic or cDNA can be used for the methods described herein.

To help overcome the moderate yields of rTE recovered by Rosenbloom and colleagues, Martin, Vrhovski and Weiss successfully synthesized and expressed a gene encoding human tropoelastin in E. Coli. In constructing the gene they tailored the rare codon bias of the synthetic sequence to match the known preferences of E. Coli. rTEtropoelastin produced by expression of synthetic genes can be used for the methods described herein.

rTE is used in the invention can be produced in non-bacterial expression vector systems. Yeast expression vector systems are well suited for expressing eukaryotic proteins and tropoelastin is a potentially excellent candidate for expression in yeast.

For large scale heterologous gene expression, the baculovirus expression vector system (BEVS) is particularly advantageous. BEVS has several advantages over other expression systems for mammalian gene expression. It is safer, easier to scale up, more accurate, produces higher expression levels, and is ideal for suspension cultures permitting the use of large-scale bioreactors. Generation of a recombinant baculovirus particle carrying a clone of elastin cDNA coding for an isoform of tropoelastin is achieved through homologous recombination or site specific transposition and is followed by recombinant baculovirus infection of insect cells (Sf9 or High Five) and subsequent recombinant gene expression as follows:

Elastin cDNA encoding tropoelastin is identified and isolated from a cDNA library. The gene is cloned into a pFastBac or pFastBac HT donor plasmid using standard restriction endonucleases and DNA ligase. Correct insertion of gene is verified by restriction endonuclease digestion and PCR analysis. The DNA is then transformed into DH10Bac cells which harbor a bacmid a mini-attTn7 target site and a helper plasmid. Once cloned into the DH10Bac cells, the elastin gene undergoes site-specific transposition into the Bacmid. Transposition results in the disruption of a LacZalpha gene and colonies containing recombinant bacmids are white. High molecular weight mini-prep DNA is prepared from selected E. Coli clones containing the recombinant bacmid and is used to transfect SF9 or High Five insect cells using CellFECTIN reagent. The insect cells produce actual baculovirus particles harboring the tropoelastin encoding gene. The virus particles are harvested and are subsequently used to infect insect cells which produce high yields of the recombinant protein product, tropoelastin.

Tropoelastin accumulated in elastin rich tissues by the inhibition of lysyl oxidase through copper deficiency or lathyrism can be isolated by exploiting tropoelastin's high solubility in short-chain alcohols. Modified methods of this alcohol extraction procedure can be used to purify rTE from expression hosts such as bacteria, yeast, insect, and mammalian cells in culture. Methods have been described in detail which involve precipitation of tropoelastin with n-propanol and n-butanol. Tropoelastin expressed in insect cells using the pFastBac HT baculovirus expression system (Life Technologies, Gaithersburg, Md.) can be purified in a single affinity chromatography step with Ni-NTA resin. The invention is not limited to any particular method of tropoelastin isolation or purification.

### Polymer Synthesis

In tissue, tropoelastin is naturally crosslinked by several tetra and bifunctional cross-links to form elastin. These crosslinks arise through the oxidative deamination and condensation of lysyl side chains. Both bifunctional lysinonorleucine and allysine aldol and tetrafunctional desmosine crosslinks are formed. Tetrafunctional desmosine crosslinks are a distinguishing feature of elastin. Tropoelastin can be converted to a tropoelastin biomaterial by oxidative deamination of lysyl residues and the subsequent crosslinking of the monomeric moiety catalyzed by the copper dependent enzyme lysyl oxidase (protein-lysine 6-oxidase).

One can crosslink tropo-elastin monomers with the same bifunctional and tetrafunctional cross-links found in elastin. However, the invention is not limited to these naturally occurring cross-links and any type of cross-link formed between tropoelastin monomers, whether produced chemically, enzymatically or radiatively, can be used for the invention.

Crosslinking tropoelastin with lysyl oxidase will produce matrices that may resemble naturally occurring ones. Lysyl oxidase (protein-lysine 6-oxidase) catalyzes the oxidation of lysine residues to a peptidyl α-aminoadipic-α-semialdehyde. This aldehyde residue spontaneously condenses with neighboring aldehydes or α-amino groups forming interchain or intrachain crosslinkages (Kagan, 1991). Lysyl oxidase from any source can be used so long as the tropoelastin it is intended to oxidize is a suitable ligand. Lysyl oxidase is typically extracted from bovine aorta and lung, human placentas, and rat lung with 4 to 6 M urea extraction buffers. Recombinantly produced lysyl oxidase can also be used to cross-link tropoelastin. Recombinant tropoelastin (rTE26A) has been cross-linked with lysyl oxidase in 0.1 M sodium borate, 0.15 M NaCl, pH 8.0 when incubated for 24 hr at 37° C. (Bedell-Hogan, 1993). Another preferred method of crosslinking tropoelastin is with γ-irradiation. γ-irradiation causes formation of free radicals which can result in crosslink formation. 20 mrad of γ-irradiation has been shown to crosslink an elastin like polypeptide, poly(GLy-Val-Gly-Val-Pro), into an elastomeric matrix and has increased the elasticity and strength of a elastin-fibrin biomaterial. The addition of chemical agents that form crosslinks when activated with irradiation can also be used. Sulfur derivatives combined with γ-irradiation been shown to further increase the strength of an elastin-fibrin biomaterial. Chemical crosslinking reagents such as glutaraldelhyde may also be used to cross-link tropoelastin matrices.

A preferred method of organizing tropoelastin monomers into fibrous structures prior to cross-linking is by taking advantage of the property of coacervation exhibited by tropoelastin. Tropoelastin is soluble in water at temperatures below 37° C., however, upon raising the temperature to 37° C. tropoelastin aggregates into a aggregated structure called a coacervate. Formation of tropoelastin coacervates may be a natural step prior to cross-link formation during elastogenesis in tissue. Coacervated tropelastin can be crosslinked by lysyl oxidase under the appropriate conditions to produce tropoelastin aggregates. Alignment may be facilitated by exposure of the tropoelastin coacervates to a magnetic field prior to crosslinking.

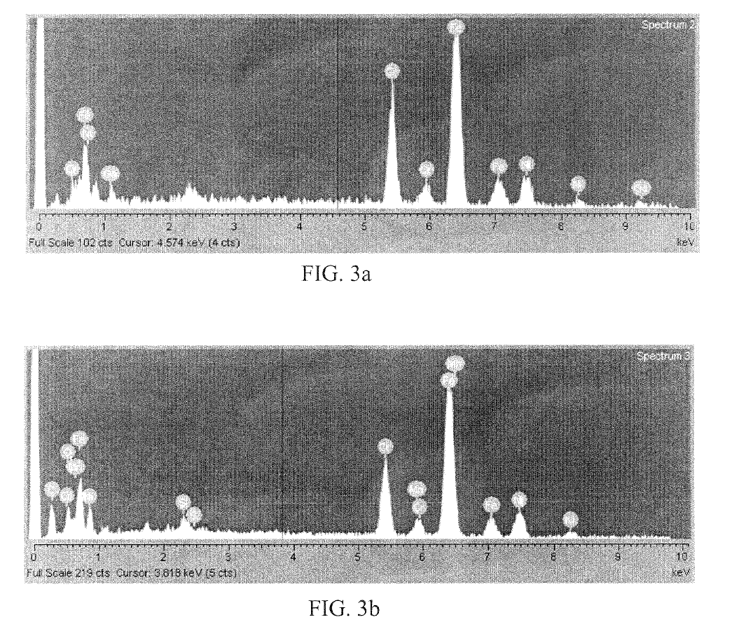

Collagen is the major structural polymer of connective tissues. Artificial collagen fibers have been produced from soluble collagen I extracts. Fibers such as these can be formed into scaffoldings onto which tropoelastin can be cross-linked into amorphous insoluble elastin producing a elastin/collagen composite (see FIG. 3). The collagen fibers lend form and tensile strength to the tropoelastin material and the crosslinked tropoelastin fibrils lend elasticity thus creating a composite material that very nearly approximates naturally occurring connective tissue.

Proteoglycans are major constituents of the extracellular matrix. The addition of Hyaluronic acid, dermatan sulfate, keratane sulfates, or Chondroitin sulfates as co-materials may further the strength and cohesion of the material. In addition, cell function is in part controlled by the extracellular matrix. Fibronectin, vitronectin, laminin nad collagen, as well as various glycosaminoglycans all mediate cell adhesion. Fibronectin has several roles in the connective tissue matrix. It has an organizing role in developing tissues and it plays a major role in cell adhesion to the extracellular matrix. Incorporation of fibronectin as a co-material may improve the cell adhesion properties of the tropoelastin based biomaterial. Microfibrils are distributed throughout the body, and are prevalent in elastic tissues and fibers. The presence of microfibrils during polymerization of tropoelastin monomers may help to organize monomers yielding a material with improved structural organization. Also, microfibrils are known to sequester calcium ions and are thought to play a role in protecting tropoelastin from chronic calcification.

### Product Synthesis

The utility of tropoelastin based biomaterials may be further improved by combining them with synthetic or natural polymer co-materials, forming composites, and by adding bioactive impregnates.

Antibiotics and/or anticoagulants or other agents can be added to the tropoelastin matrix providing localized drug therapy and preventing infection. In surgical repair of abdominal traumatic injuries, infection represents a major problem particularly when vascular prosthetic implants are used. An tropoelastin graft with antibiotic incorporation may be ideal because it avoids sacrifice of an autologous artery or vein which decreases surgical time and precludes the necessity to use synthetic prosthetic materials which may be more prone to infection than tropoelastin grafts. Bioactive impregnates may also include anti-coagulants (Hirudin), coagulants, anti-proliferative drugs (Methatrexate), growth factors, anti-virals, and anti-neoplastics.

For delivery of biomaterial in the form of an intravascular stent, the biomaterial can be pre-mounted upon a deflated balloon catheter. The balloon catheter can be maneuvered into the desired arterial or venous location using standard techniques. The balloon can then be inflated, compressing the stent (tropoelastin biomaterial) against the vessel wall and then laser light delivered through the balloon to seal the stent in place (the dye can be present on the outside of the biomaterial). The balloon can then be deflated and removed leaving the stent in place. A protective sleeve (of plastic or the like) can be used to protect the stent during its passage to the vessel and then withdrawn once the stent is in the desired location.

The biomaterial of the invention can also be used as a biocompatible covering for a metal or synthetic scaffold or stent. In such cases, simple mechanical deployment can be used without the necessity for laser bonding. Laser bonding can be employed, however, depending upon specific demands, eg, where inadequate mechanical bonding occurs, such as in stent deployment for abdominal aortic aneurysms. An alternative catheter-based vascular stent deployment strategy employs a temporary mechanical stent with or without a balloon delivery device.

A further catheter-based vascular stent deployment strategy employs a heat deformable metal (such as nitinol or other similar type metal) scaffold or stent or coating that is incorporated into the catheter tubing beneath the stent biomaterial. The stent is maneuvered into the desired location whereupon the deformable metal of the stent is activated such that it apposes the stent against the vessel wall. Laser light is then delivered via an optical fiber based system, also incorporated into the catheter assembly.

The biomaterial can include antibiotics, coagulants or other (drugs desirable for various treatments that provide high local concentrations with minimal systemic drug levels.

For certain applications, it may be desirable to use the biomaterial of the invention in combination with a supporting material having strong mechanical properties. For those applications, the biomaterial can be coated on the supporting material (see foregoing stent description), for example, using the molding techniques described herein. Suitable supporting materials include polymers, such as woven polyethylene terepthalate (Dacron), teflon, polyolefin copolymer, polyurethane polyvinyl alcohol or other polymer. In addition, a polymer that is a hybrid between a natural polymer, such as fibrin and elastin, and a non-natural polymer such as a polyurethane, polyacrylic acid or polyvinyl alcohol can be used (see Giusti et al, Trends in Polymer Science 1:261 (1993). Such a hybrid material has the advantageous mechanical properties of the polymer and the desired biocompatibility of the tropoelastin material. Examples of other prostheses that can be made from synthetics (or metals coated with the tropoelastin based biomaterial or from the biomaterial/synthetic hybrids include cardiac valve rings and esophageal stents.

The tropoelastin-based prostheses of the invention can be prepared so as to include drug; that can be delivered, via the prostheses, to particular body sites. For example, vascular stents can be produced so as to include drugs that prevent coagulation, such as heparin, or antiplatelet drugs such as hirudin, drugs to prevent smooth muscle ingrowth or drugs to stimulate endothelial damaged esophageal segments during or following surgery or chemotherapy for esophageal carcinoma or endothelial regrowth. Vasodilators can also be included.

Prostheses formed from the tropoelastin bio-material can also be coated with viable cells, cells from the recipient of the prosthetic device. Endothelial cells, preferably autologous (eg harvested during liposuction), can be seeded onto the elastin bioprosthesis prior to implantation (eg for vascular stent indications). Alternatively, the tropoelastin biomaterial can be used as a skin replacement or repair media where cultured skin cells can be placed on the biomaterial prior to implantation. Skin cells can thus be used to coat elastin biomaterial.

All documents cited above are hereby incorporated in their entirety by reference.

A dependable expression system to produce recombinant human tropoelastin has been established as hereinafter described. A purification procedure has been developed that results in a >95% pure product. Tropoelastin has been cross-linked with a chemical agent to form mature elastin, demonstrating that the recombinant tropoelastin has the biochemical properties necessary to form a structured biopolymer. E. coli cell lines that express recombinant human lysyloxidase that is the natural initiator of cross-link formation in tissues have also been created.

An increase in the yield of recombinant tropoelastin from our E. coli expression system. A continuous production of recombinant human tropoelastin using 10 liter a bioreactor can be provided. Cultures of E. coli have been developed to produce up to 4 gm of human tropoelastin in one 10-litre batch culture. This has been made possible primarily due to the use of a bioreactor and a codon-use optimized E. coli synthetic tropoelastin gene. Yeast extract and tryptone have been removed from the cell culture medium so that a chemically defined medium is formed. The product is retained within the E. coli that is harvested by centrifugation. Approximately 300-350 gm of E. coli wet pellet (biomass) is collected. A 10-fold increase in yield is provided when the new tropoelastin gene was used. These data also show that increasing the inducer IPTG concentration increases the yield of tropoelastin but decreasing the temperature at induction reduces the yield. The assay for tropoelastin is based upon the quantitation of stained protein bands in SDS polyacrylamide electrophoresis gels.

The biomass from the bioreactor, which contains the tropoelastin, can be collected by centrifugation weighed and suspended in 70% formic acid (typically 150 gm in 300 ml). Cyanogen bromide (10% w/w) is added and the mixture stirred at room temperature for 5 hours by which time a clear pale yellow solution is formed. The cyanogen bromide is removed in vacuo and the sample reduced to half its volume. The sample is dialyzed against 0.1% trifluoroacetic acid (4×4 liters) at 4° C. Insoluble material is removed by centrifugation and the supernatant lyophilized. This material (8-10 gm) is dissolved in a 25 mM K2HPO4 buffer pH 7.5 containing 6M urea, and applied to a column (5×22 cm) of BioRad HS50 cation exchanger. The sample is eluted with a 3-step elution at 0.05M, 0.25M and 0.5M NaCe. The middle fraction which contains the tropoelastin was dialysed into 0.1% trifluoroacetic acid and applied to a reversed phase column (Vydac C4 21×25 mm) and eluted at room temperature with an acetonitril gradient (0-30%). Tropoelastin containing fractions are pooled, lyophilized and applied to a second cation exchange column (2.5×22 cm) of SP Sepharose (Amersham Biochemicals) equilibrated with 25 mM sodium acetate buffer pH 5.0 containing 6M urea. The sample is eluted with a linear gradient of NaCl from 0 to 0.1 M. Tropoelastin containing fractions are pooled, desalted by dialyzing against 0.1% trifluoroacetic acid and lyophilized. The final human tropoelastin product is 95+% pure and will be improved, but is sufficiently pure for cross-linking studies and mechanical testing (FIG. 16).

Lysyl oxidase can be used to cross-link the tropoelastin coacervates, but other chemical reagents can be used. Tropoelastin molecules can be pre-aligned for cross-link formation to take place. This can be achieved by warming the sample at a controlled rate to coacervate the tropoelastin molecules causing them to associate and form a viscous phase that can be collected by centrifugation. This process can be followed spectrophotometrically, the rate and extent of coacervation being an indicator of tropoelastin quality and characteristic for the isoform being used. A chemical cross-linking reagent di-(sulfo-succinimide) suberate was tested because it has two important characteristics for use in biological systems. First, it is water-soluble which is important for reaction with proteins under physiological conditions. Second, when incorporated into protein the cross-link structure is —(CH2)6- which would not be expected to cause a biological response when the biopolymer is implanted into living tissues. In an experiment, sodium di-(sulfo-succinimide) suberate was dissolved in dimethyl sulphoxide and mixed with tropoelastin coacervate (˜100 μl) on ice for 15 minutes, and then left at room temperature overnight. A white solid material was formed which was collected by centrifugation, washed with water to remove reagents, with 6M urea to remove uncross-linked tropoelastin, and again with water to remove urea. The polymer had the consistency of rubber and appeared to be elastic. These are desired properties, which will be quantitatively characterized. A technical problem that had to be resolved concerns mixing the tropoelastin coacervate, which is a viscous solution, with cross-linker solution fast enough to give a homogeneous phase before cross-linking takes place. Slowing down the reaction rate by reducing the concentration of cross-linker is one possibility but this produced a product that was not fully cross-linked. However, we could correct this by soaking the product in a cross-linking solution to complete the reaction. Another possibility being investigated is to carry out the cross-linking reaction at a sub-optimal pH and low temperatures to slow the reaction rate. Static mixers may achieve high speed mixing. 4 cm×6 cm patches of human elastin can be fabricated approximately 1 mm thick. This initially requires a solution containing 1.5 gm of tropoelastin. The solution is warmed to 37° C. to coacervate the tropoelastin. The coacervate is a viscous liquid and forms a separate phase that can be collected by centrifugation. The coacervate is mixed at −10° C. with a bifunctional crosslinker and poured into a mould. The mould is warmed to 37° C. and held at that temperature in an oven overnight. The elastin patch is removed from the mold and washed with 6M guanidine hydrochloride to remove unreacted, or uncrosslinked components. The patch is then re-equilibrated in PBS for testing. The mechanical properties of the human synthetic elastin polymer are compared to those of natural elastin prepared by extracting swine aorta. Stress/strain curves indicate that the human elastin (tropoe) compares favorably with natural aortic elastin H1 but is somewhat weaker. The tropoelastin-derived patches have a mesh-like structure with large pores as shown by the scanning electron microscopy imaging. This structure will be advantageous for cell penetration and the reinforcement of the structure with a natural collagenous matrix in vivo. However, to increase their strength, the weight of Tropoelastin per patch must be increased and the pores decreased in size. There is a limit to the concentration of the tropoelastin in the solutions used to make patches. Forming patches may be accomplished under centrifugal force. In order to do this, a low speed centrifuge with a swing-out rotor is employed. The tropoelastin solution and cross-linker will be mixed at low temperature, poured into a mold in the centrifuge, the centrifuge started and the temperature increased to 37° C. to coacervate the tropoelastin.

For vascular repair, tubular metal stents have been an important component in the spectrum of technologies available to the surgeon repairing vascular injuries. The major limitation of present technologies is inherent to the metals themselves-both being foreign bodies readily identifiable to the immune system and for the fact that they are inherently thrombogenic. Because of these limitations, stents are only useful for larger vessels and even the most modern metal vascular stents that elute anti-inflammatory and other drugs from their surfaces, thrombosis is a concern that may be present for many years. In the case of late stent thrombosis, the recent mortality rate is 45%. With over one million drug eluting stents implanted in mostly civilian patients world wide and at least a 1% incidence of late stent thrombosis-a significant and deadly new problem is emerging.

To establish the biocompatibility, thrombogenicity and proof of principle of placing a recombinant human elastin coating on medical stainless steel stents to improve their biocompatibility and utility we coated AVE-Medtronic commercially available stainless steel stents 3 mm diameter×12 mm length with rTPE and cross-liked it in. This process yielded a stable, uniform, covalently bound elastin [show picture]. It has been established that the coating was stable when placed in 3 mm diameter swine coronary artery using conventional balloon deployment devices. Scanning EM after 2 hours of implantation showed no evidence of coating disruption. Fibrin or clot adherence was minimal and not different than an identical uncoated stent placed in the other coronary artery. Late thrombosis of DES may be due to synthetic polymer coatings. Elastin is a flexible, biocompatible, non-thrombogenic protein that inhibits smooth muscle migration and can also bind drugs. Human recombinant elastin (HRE) covalently bounded coatings on metal stents compared to bare metal stents (BMS) in a randomized, double blind study to compare thrombosis, thrombus adherence, inflammatory response and neointimal hyperplasia in swine coronary arteries.

46 anesthetized 40 kg swine were pretreated with oral aspirin (ASA) 325 mg and Clopidogrel 75 mg, and heparin (100 IU/kg) to ACT>250. Medtronic AVE S7 stents (3.00 mm×12 mm), uncoated or with 3 μm (HRE) coatings were placed randomly, in the LAD or LCX coronary arteries and blinded to stent type. Clopidegrel and ASA were given orally until angiography, sacrifice and perfusion fixation at 2 hours, 7, 14 and 28 days. All data were analyzed by an independent observer blinded to stent type.

There were no acute thrombotic events or angiographic restenosis >20% in either group. At 2 hours there was no significant difference in thrombus adherence or coating disruption by scanning EM. Fibrin amount was reduced by HRE-1.22±0.54 vrs BMS-2.00, p=0.009, and % of struts with fibrin attached was reduced by HRE 23.43±9.57 vrs BMS-90.2±7.70, p=0.017 at 7 and 14 days and equivalent at 28 days. Inflammatory scores, % endothelialization, % stenosis, and neointimal thickness or area were not significantly different between BMS and HRE coated stents.

Human recombinant elastin coatings on metal stents reduced thrombus adherence and amount compared to uncoated metal stents. HRE coatings appeared biocompatible without evidence of increased inflammation, neointimal hyperplasia, or allergic eosinophilic reaction even with the cross-species vascular exposure. Elastin with its inherent ability to reduce smooth muscle cell migration and bind drugs such as sirolimus may be an excellent physiologic coating for vascular stents and has the potential to reduce thrombosis or long term adverse responses to synthetic stent coating materials.

It has been demonstrated that the recombinant human elastin coating was superior to the conventional medical stainless steel stent and may solve one of the most important problems in this field-thrombosis. The rTPE coating did not, however, diminish the inflammatory response to metal stents and all measures of inflammation or intimal hyperplastic response were not significantly different. While this finding may diminish the promise of a more biocompatible tissue interface for the metal, it is very likely that part of this inflammatory response may be due to the fact that this is a human protein placed on the inner blood flowing surface of the swine artery and may be a modest inflammatory response to a cross species protein implant. The lack of a severe inflammatory response to a foreign protein may attest to the immune poor quality of intact elastin proteins. It may be then that metal stents coated with porcine elastin in the swine model may have the optimal response and be more reflective as an animal model of the human protein placed in humans.

### Cloning of Human ELN cDNA

A human fetal heart cDNA library (Clontech, Palo Alto Calif.) was screened with a human elastin gene (ELN) specific probe using standard methods.

Approximately 1×106 clones were screened with a 175 bp PCR fragment of human elastin cDNA encompassing exon 20. The screening yielded 85 positive plaques. Isolated positive clones were further screened by PCR for the presence of the 5γ and 3γ UTRs to identify full-length clones. Clones that contained full-length transcripts were purified to homogeneity and subcloned into pLITMUS 29 (New England Biolabs) and sequenced with pUC19/M13 forward and reverse primers as well as six internal elastin cDNA-specific sequencing primers to determine isoform composition. Fifteen tropoelastin full-length clones were sequenced, representing nine different splice variants. The most abundant splice variant found in vascular tissue was selected as the template for recombinant elastin production. The composition of this splice variant includes all coding exons except for exons 22 and 26A. These rarely utilized exons are seldom included in ELN mRNA. The selected tropoelastin cDNA was engineered to remove exon 1, which encodes the secretion signal sequence and would not be recognized and cleaved by E. coli. Removal of exon 1 prevents the secretion signal sequence from erroneously being incorporated into the tropoelastin molecule. A methionine residue was added to the 5γ end of exon 2. The methionine residue separates the GST fusion protein from the amino-terminus of tropoelastin. This provides a cyanogen bromide cleavage point to facilitate purification. Since there are no other methionine residues in tropoelastin, the final product is unaffected by treatment with cyanogen bromide, but other contaminating proteins are cleaved simplifying their removal from the final product. The altered insert was cloned into pGEX2T (Amersham Biosciences), which produces a glutathione-S-transferase (GST) fusion protein with an amino-terminal GST tag. The construct was transfected into E. coli BL21 Codon Plus cells (Stratagene) for recombinant protein expression.

There is significant codon bias for tRNAs expressed by E. coli compared to those expressed by human cells, so a synthetic tropoelastin gene was made that produces the identical protein to the clone described above but using codons that are commonly used by E. coli. The synthesized insert (Entelechon GmbH, Germany) was cloned into the modified pGEX2T vector and transfected into E. coli BL21 cells (Stratagene) for recombinant protein expression. The new construct was expressed in E. coli grown in shaker flasks and compared to the expression levels obtained from the original human clone. A 3 to 5 fold increase in tropoelastin production was achieved. Frozen stocks of the E. coli containing the optimized sequence were prepared and used to seed a 10-liter bioreactor for routine production of tropoelastin.

### Tropoelastin-Coated Stents

Stainless steel stents have been modified to allow for covalent attachment of tropoelastin. The surface is first oxidized electrochemically, silanes with amine termini are attached to the surface, the stent is dipped into tropoelastin coacervate, and finally the tropoelastin is crosslinked into a polymeric material bound to the stent surface. Microscopic inspection of the stents indicates smooth and continuous coating. The coating is flexible and remains intact after expansion of the stents and after γ-irradiation. Biological testing is just beginning.

### EXPERIMENTAL

Toluene, acetone, isopropyl alcohol, ethyl acetate, and bis(N-hydroxylsuccinimide ester) were purchased from Sigma-Aldrich and used without further purification. (3-Aminopropyl)triethoxysilane (APS) was from TCI America. Stainless steel plate (type 302) for preliminary studies of tropoelastin coating was obtained from AISI (American Iron and Steel Institute). Stainless steel stents (AVE Medtronic S7, 3 mm diameter, 12 mm length) were used for implantation study as provided. Tropoelastin was provided from Oregon Medical Laser Center, Portland, Oreg. All equipment and glassware were sterilized with steam or sterrad.

### Instrumentation

Electrochemical experiments were carried out with a model 273 potentiostat/galvanostat controlled by M270 software (EG&G, Princeton, N.J., USA). A conventional three-electrode cell was used, including a Pt wire (Aldrich) as a counterelectrode, a stent or a stainless chip as a working electrode, and a reference electrode of Ag/AgCl in saturated KCl.

X-ray photoelectron spectroscopy (XPS) measurements were performed with a Kratos Hsi XPS instrument using a monochromatic A1 source (operated at 200 W). Scanning electron microscopy (SEM) was carried out using FEI Siron SEM, which was equipped with energy dispersive X-ray (EDX). All samples were coated with gold before scanning. Implanted samples were rinsed with saline solution three times, then once with distilled water, dried, and finally coated with gold.

Atomic force microscopy (AFM) for surface analysis of coated samples was performed with a Nanoscope IIIA (Veeco, Santa Barbara, Calif.) using a 125 μm cantilever equipped with a silicon nitride tip in the tapping mode at an oscillating frequency of 300 kHz.

### Methods

The entire coating procedure for samples to be implanted was performed in a clean room. Stainless steel foil was cut into 1.0×1.5 cm samples, sonicated in aqueous detergent solution for 30 min, followed by sonication in 1:1 acetone/isopropyl alcohol solution for 30 min, then dried in an oven for 6 hours at 70° C. Initially the sample was cathodically polarized at −0.60 V for 15 min and then pulsed to +0.25 V for 1 min. After the oxidation, the sample was washed with sterile distilled water and dried for 6 hours at 70° C. The oxidation process was intended to form a surface oxide layer, expected to be more favorable for subsequent binding of the silane derivatives. The oxidized samples were treated with (3-aminopropyl)triethoxysilane (APS) (5 μL of APS dissolved in 10 mL of toluene) and allowed to react for 24 hours. They were then placed in fresh toluene and sonicated for 10 min to remove excess material not tightly bound, washed with toluene three times, and heated at 105° C. for 10 min. The purpose of this silanization treatment is to generate free primary amines on the surface, which are expected to react chemically like the lysine residues in tropoelastin, enhancing the binding between the surface and the crosslinked tropoelastin.

Fe-(oxidation)->Fe—O-(APS)->Fe—O—Si—CH₂—CH₂—CH₂—NH₂

A solution of tropoelastin in phosphate buffer solution (pH 7.4) was warmed to 37° to allow coacervation. The silanized stainless steel chip or stent was dipped into this coacervate for 5 min and withdrawn. These coacervate-coated samples were centrifuged at 1,000 rpm to remove excess material. The coacervate-coated stent was then dipped into a solution of bis(N-hydroxysuccinimide ester), a crosslinking reagent (10 mg), dissolved in ethyl acetate (10 mL) overnight. The use of a water-immiscible solvent like ethyl acetate minimizes redissolving of the coacervate. The crosslinked tropoelastin-coated samples were rinsed carefully with pure ethyl acetate three times and air-dried for 24 hours. The final stainless steel chips were used for surface analysis and the stents were mounted on a balloon deployment device, inserted in a sterile bag, and sent for γ-irradiation sterilization. Both coated and non-coated stents were deployed in the subsequent biological studies.

### Results

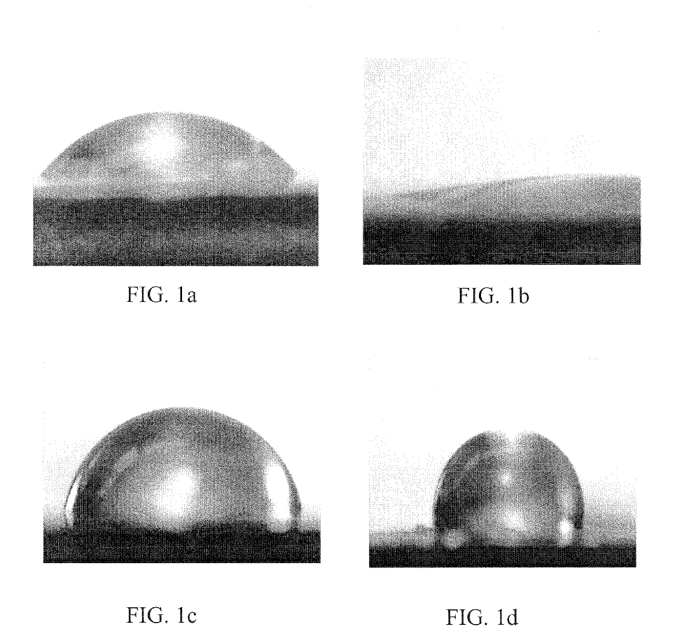

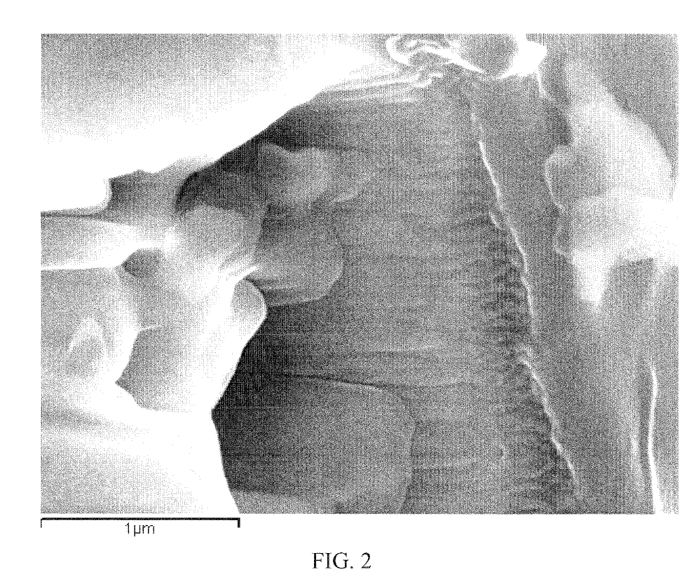

FIGS. 1(a)-(d) depict the contact angles of water on a substrate, more particularly, a flat stainless steel surface. FIG. 1(a) is an unpretreated stainless steel substrate (contact angle=60°). FIG. 2(b) is a pretreated (oxidized) stainless steel substrate (contact angle=12°). FIG. 1(c) is a pretreated substrate coated with a biocompatible intermediate (silanized) bonding layer (contact angle=81°). FIG. 1(d) is pretreated substrate coated with a tropoelastin polymer (contact angle=121°).

Contact angle measurements indicate the wetting properties of a surface, typically interpreted as hydrophilicity or hydrophobicity. Measurements were performed by carefully placing a 2 μL drop of distilled water on a horizontal surface and visually observing and measuring the angle made at the liquid/solid interface. The original stainless steel shows a contact angle of 60°. After oxidation, the contact angle is much lower (12°), indicating that the stainless steel surface is substantially more hydrophilic (polar), indicating the expected change upon oxidation. After silane treatment of the freshly oxidized surface, the contact angle is much higher (81°), higher even than the original stainless steel, indicating that the surface is substantially more hydrophobic (nonpolar). After coating with the coacervate, the contact angle is very high (121°), consistent with the known hydrophobicity of tropoelastin. To confirm that the silane-treated surface contained amine groups, contact angles were measured using drops of buffered solutions rather than pure water. The contact angle at pH 10 was unchanged, but at pH 3, 4, or 5, the contact angle was distinctly lower (60°), consistent FIGS with protonation of amines.

Energy dispersive X-ray analysis (EDX) is a technique that detects specific elements at the surface of a sample. A tropoelastin-coated stent sample was cut to observe the cross section by using focus ion beam (FIB) as illustrated in FIG. 2. The tropoelastin-coated side, metal side, and interface between metal and polymer side were observed with EDX. Silicon was detected at 1.75 keV, which indicates that surface modification with APS was successfully performed. A strong carbon band was observed on polymer side and interface indicating the existence of polymer, which was rarely observed on metal side. Less metal energy intensity bands were observed with tropoelastin-coated side compared to metal side and interface.

FIG. 2 are cross-sectional SEM images of tropoelastin-coated stent: tropoelastin film side (spectrum 1); stainless steel side (spectrum 2); interface area between metal and tropoelastin film (spectrum 3). FIG. 3 are EDX spectra of tropoelastin film side (spectrum 1); stainless steel side (spectrum 2); interface area between metal and tropoelastin film (spectrum 3).

X-ray Photoelectron Spectroscopy (XPS) is a technique that detects specific elements at the surface of a sample. Table 1 describes the surface composition of each sample. Silanized sample showed the existence of silicon and nitrogen, which indicates the existence of APS molecule on the surface of silanized stainless steel sample. The carbon peak was analyzed in more detail.

TABLE 1
Surface composition derived from XPS analysis

| Sample | C | O | N | Si | Fe | Other |
|---|---|---|---|---|---|---|
| Stainless steel | 31 | 54 | | | 13 | 2 |
| Silanized | 54 | 24 | 7 | 9 | 6 | |
| Tropoelastin | 62 | 16 | 16 | | | 6 |

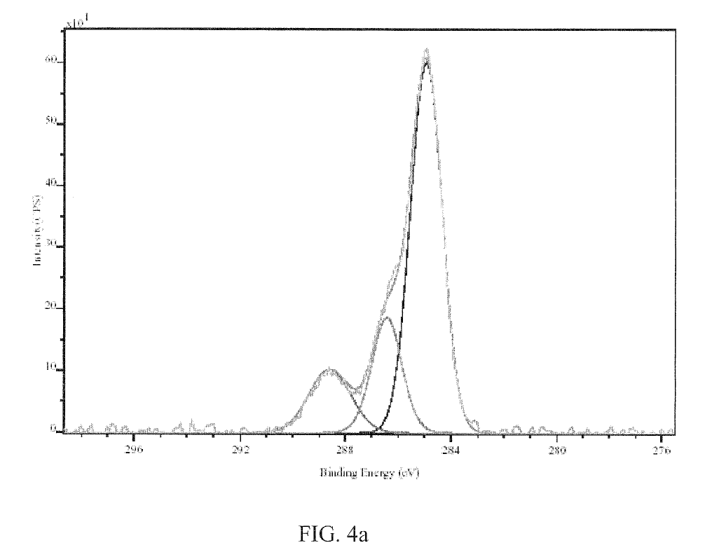

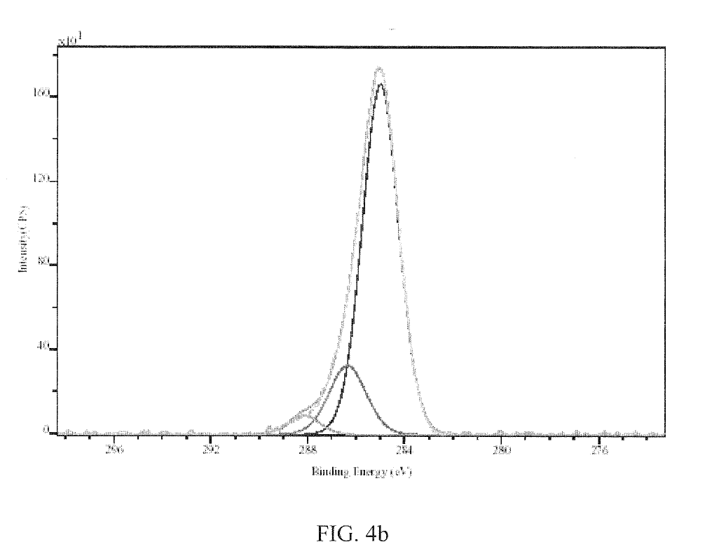

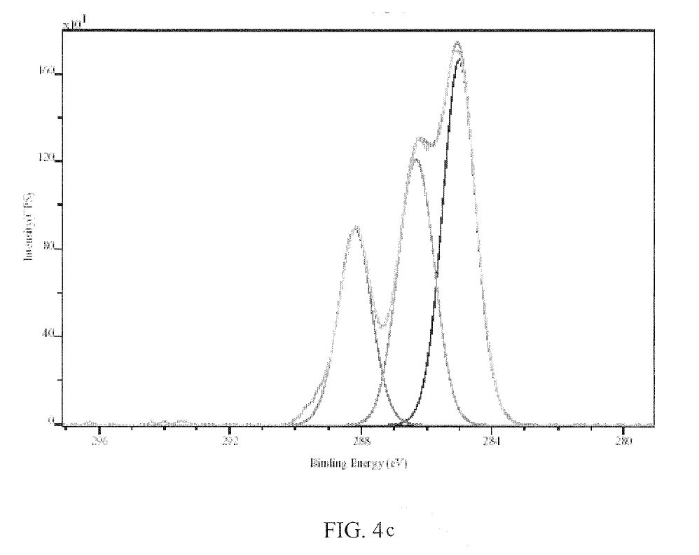

FIGS. 4(a)-(c) are C1s XPS spectra. FIG. 4(a) is a bare stainless steel, FIG. 4(b) is an intermediately coated (silanized) stainless steel substrate, and FIG. 4(c) is a tropoelastin-coated stainless steel substrate. FIGS. 4(a)-(c) shows C1s photoelectron spectra for the bare stainless steel, a silanized sample, and a tropoelastin-coated stainless steel chip. Binding energy at 285.0 eV is analyzed to be hydrocarbon, at 286.5 eV to be carbon in C—O and C—N bonds, and at 288.6 eV to be carbonyl (amide) carbon. Bare stainless steel sample shows high intensity of hydrocarbon, which is usual for the sample exposed to air contaminants (FIG. 4(a)). The intensity at 286.5 eV from silanized sample was about double that of the original stainless steel, indicating the existence of amine groups (FIG. 4(b)). The tropoelastin-coated stainless steel sample (FIG. 4(c)) showed much higher intensities indicating carbon bound to N and O.

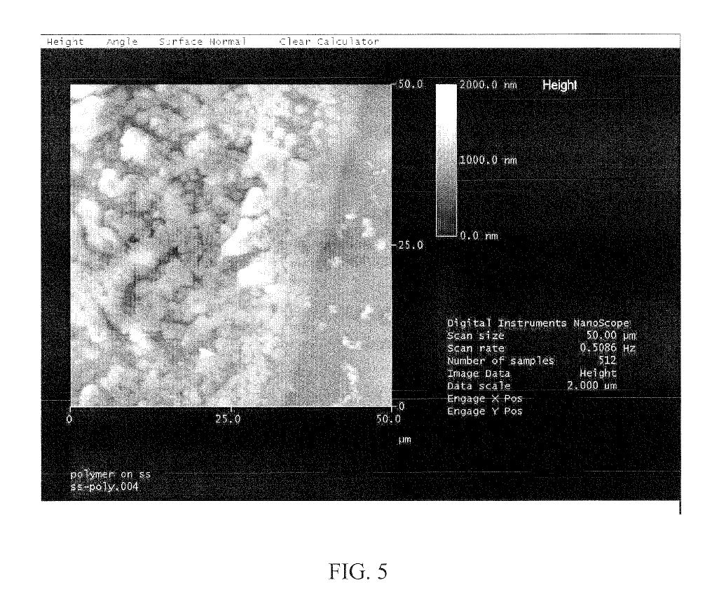

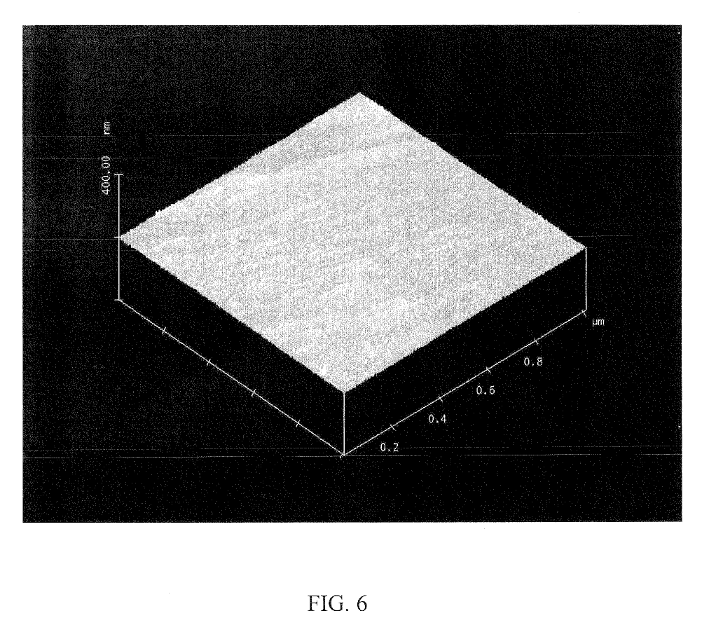

FIG. 5 depicts atomic force microscope (AFM) images. FIG. 5(a) is a crosslinked tropoelastin-coated stainless steel (50 μm full scale) with the uncoated surface on the right side. FIG. 5 (b) shows surface features of a coacervated coating (5 μm full scale). Atomic force microscopy was used to detect surface features of the coated and crosslinked tropoelastin film on stainless steel samples. AFM images of tropoelastin-coated stainless steel chip are described in FIGS. 5(a)-(b). Surface feature of coacervated coating was observed (FIG. 5(b)). As a means of creating thinner and more continuous films on the stents, after each dip-coating step, the stent was subjected to centrifugal spinning (1000 rpm for 5 min) to remove extra material from the surface of stent and more evenly distribute the viscous coacervate.

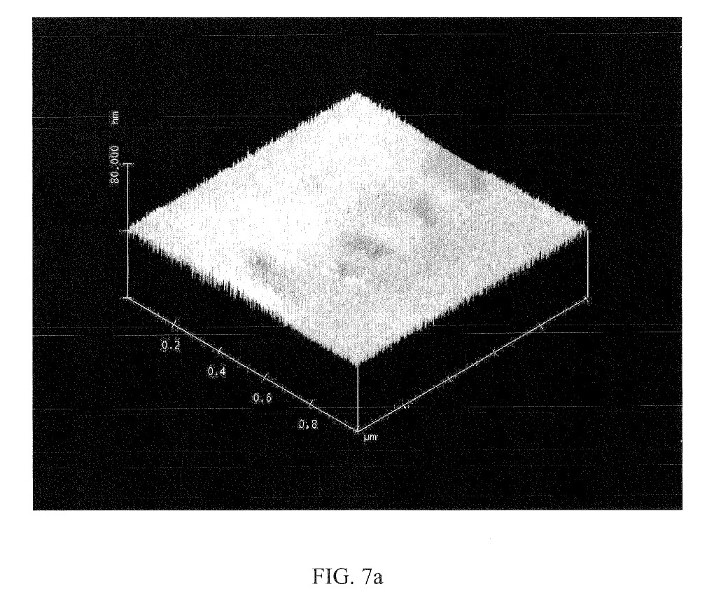

FIG. 6. are atomic force microscope (AFM) images of (a) an uncoated stent (x, y dimensions 1 μm full scale, z-axis=400 nm/div) and (b) a centrifugally treated dip-coated stent (x, y dimensions 1 μm full scale, z-axis=100 nm/div). AFM images of a centrifugally treated dip-coated stent illustrates the relative smoothness of the surface even on a submicrometer scale.

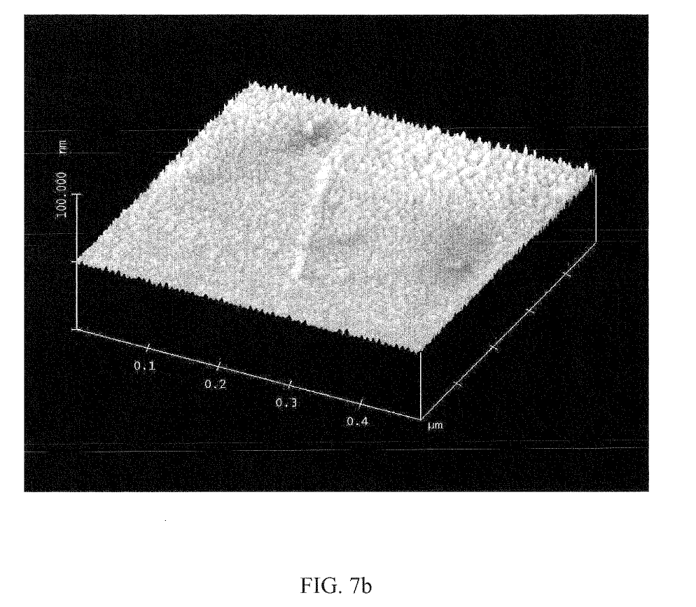

FIG. 7 are tomic force microscope (AFM) images of the inside of a centrifugally treated dip-coated stent ((a) x, y dimensions 1 μm full scale, z-axis 80 nm/div) and the outside surface ((b)−x, y dimensions 0.5 μm full scale, z-axis=100 nm/div). Both the outer and inner surfaces of the stent were examined.

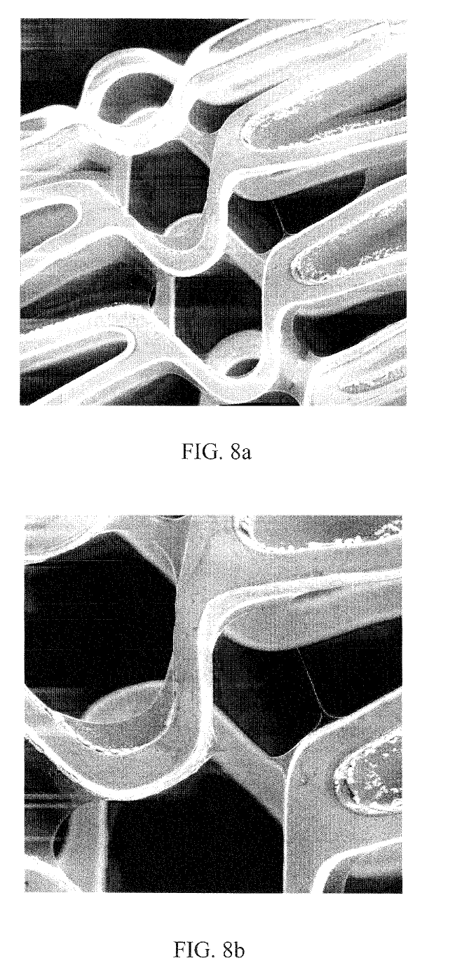

FIG. 8 shows scanning electron microscope (SEM) images of a dip-coated and crosslinked stent. Relatively thick film material can be seen in the curves of the stent. Extra material was observed from SEM images after manual spinning (FIG. 8).

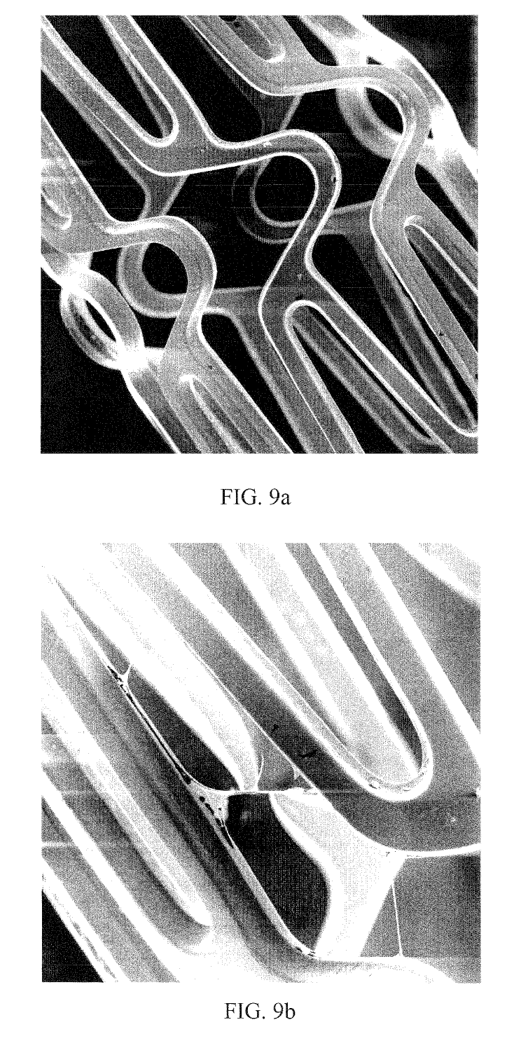

FIG. 9 shows an SEM image of a dip-coated and crosslinked stent that had been treated centrifugally. Centrifugal spinning removes all extra material as shown in FIG. 9. A coated stent was expanded in water to imitate a biological testing situation.

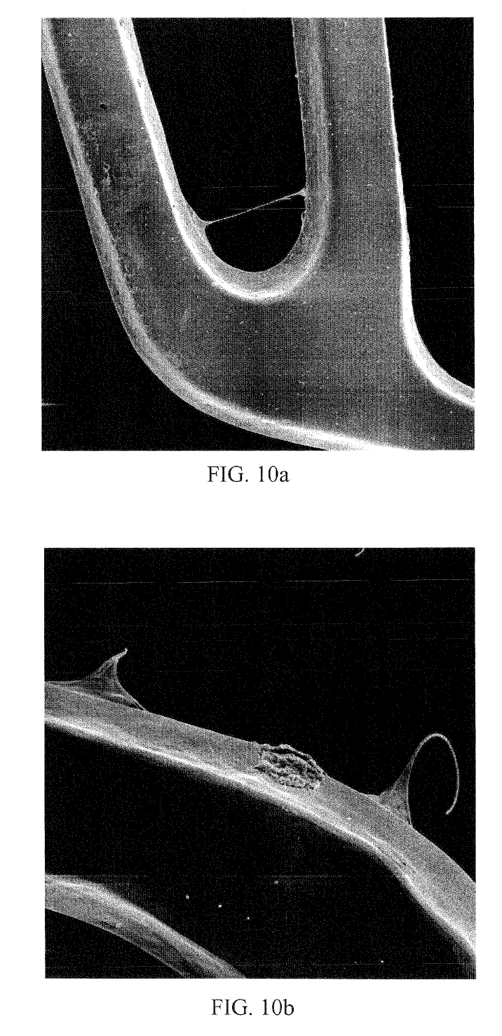

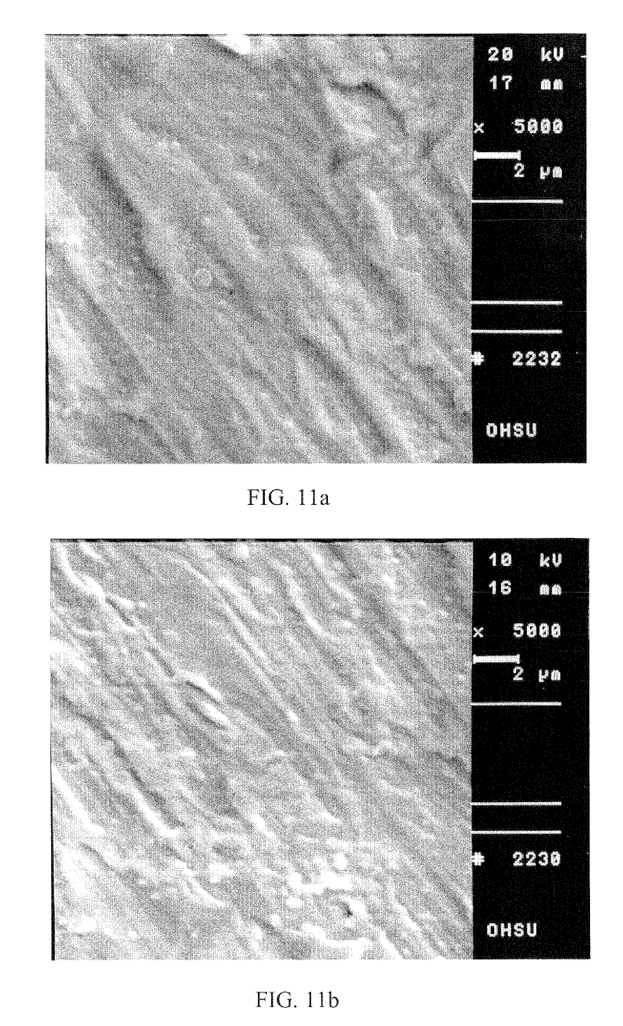

FIG. 10 is SEM images of a centrifugally treated dip-coated stent before and after expansion under water. After expansion the coating appeared to remain intact (FIG. 10). Effect of γ-Irradiation for sterilization was examined with SEM.

FIG. 11 are SEM images of the surface of an expanded and γ-irradiated coated stent. No minor effect was observed from SEM images of tropoelastin-coated surface after γ-Irradiation.

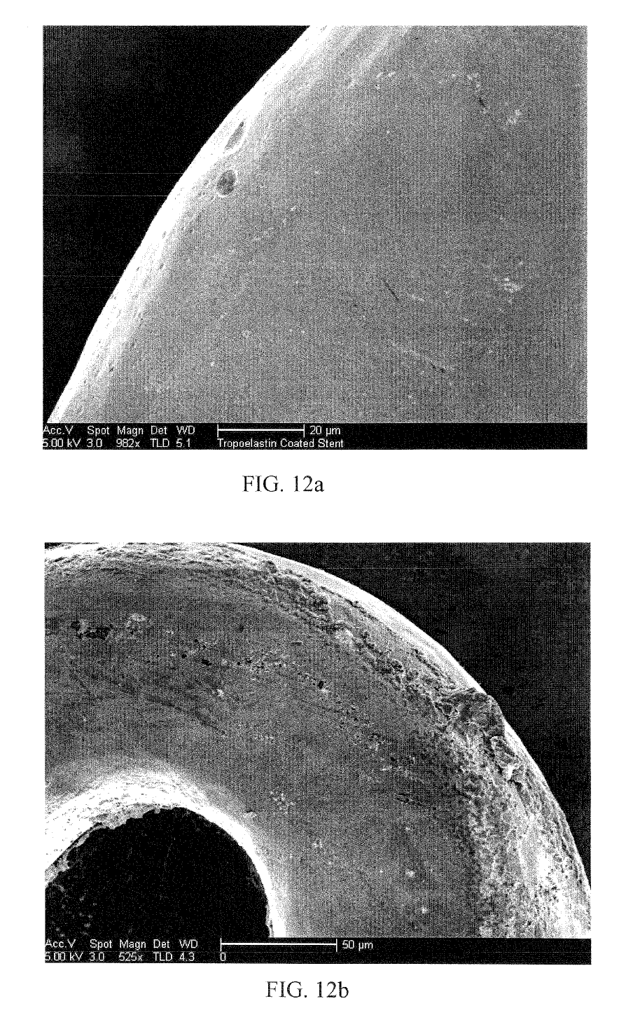

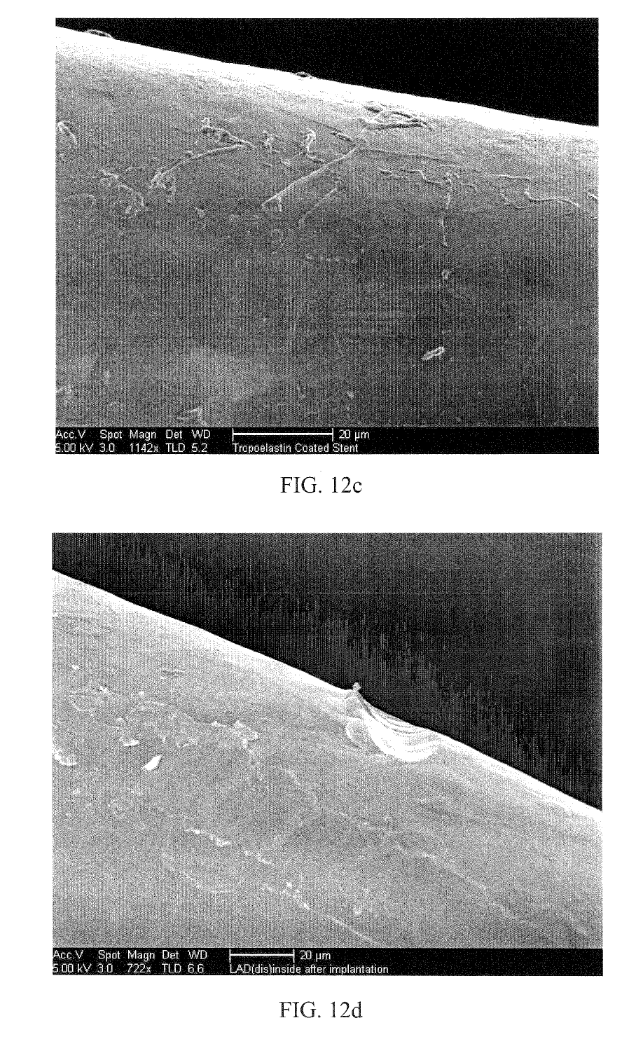

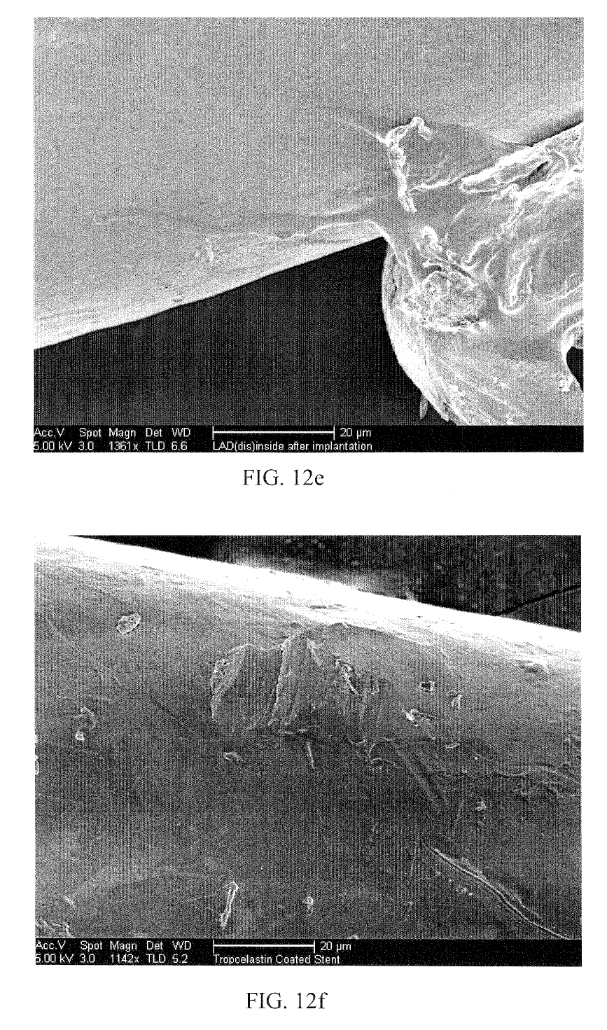

FIG. 12(a)-(f) are SEM images of (a) uncoated stent, (b) crosslinked tropoelastin-coated stent before implantation, and (c-f) coated stents after two hours implantation. AVE Medtronic S7 stents (3 mm diameter, 12 mm length, round cross-section) were chosen to produce smooth and uniform coating on entire surface for samples to be implanted. FIG. 12(a) illustrates the surface features of bare stents, which includes small pits on the surface. These features were entirely covered after the tropoelastin coating, as shown in FIG. 12(b). After implantation (FIGS. 12(c-f), some biological fibers (FIG. 12(c)) and biological adhesion (FIG. 12(e)) were observed after two hours of implantation.

### In Vivo Implant Method

Forty-three stents were implanted into the coronary arteries of domestic swines. The stented vessels were dissected at the sponsor facilities and sent to CV Path for histology processing. Twenty-three vessels were implanted with covalently bound human recombinant elastin (HRC) metal stent coating (5 μm thickness) and twenty vessels were randomly implanted into LAD or LCX arteries with bare metal stents (BMS) uncoated 3 mm×12 mm Medtronic-AVE S7 stents. The animals were survived for 7-days (HRC n=6 and BMS n=6), 14-days (HRC n=6 and BMS n=6), and 28 days (HRC n=8 and BMS n=7). One animal (#489) was survived for 60 days ((HRC n=1 and BMS n=1). All stented vessels were radiographed at CV Path to locate and assess stent placement. For light microscopy processing, the stented vessel segments were dehydrated in a graded series of ethanol and embedded in methylmethacrylate plastic. After polymerization, two to three millimeter sections were sawed from the proximal, mid and distal portions of each stent. Sections from the stents were cut on a rotary microtome at four to five microns, mounted and stained with hematoxylin and eosin and elastic Van Gieson stains. All sections were examined by light microscopy for the presence of inflammation, thrombus, neointimal formation, and endothelialization and vessel wall injury.

All procedures of handing and caring for the animals were performed in accordance with the 1996 National Research Council “Guide for the Care and Use of Laboratory Animal” and approved by the Institutional Animal Care and Use Committee of the Legacy Clinical Research and Technology Center of the Legacy Health System, Portland, Oreg. and the United States Army Medical Research and Material Command Animal Care & Use Office.

Domestic swine, 40.6 kg (±4.60, with the range being 34.3-52.7 kg) were pretreated with Aspirin 325 mg, Nifedipine XL 30 mg (UDL Laboratories Inc., Rockford, Ill.) and Plavix 150 mg (Bristol-Meyers-Squib/Sanofi Pharmaceuticals, New York, N.Y.) the day before surgery. All animals were fasted the evening prior with water allowed ad libitum. The day of surgery they were given Aspirin, 325 mg, and Plavix 150 mg. An intramuscular injection of tiletamine/zolazepam mixture, 4-9 mg/kg (Telazol®, Fort Dodge Laboratories, Fort Dodge, Iowa) was given, as well as Atropine 0.06 mg/kg (Phoenix Scientific, St. Joseph, Mo.). Mask induction was performed with Isoflurane, 5%, in oxygen. Oral intubation took place followed by mechanical ventilation, with Isoflurane continued at 2-3%. The swine were placed in a dorsal recumbent position and the medial thighs clipped, then prepped and draped in a sterile fashion. A right femoral artery cutdown was performed and a 7fr sheath introduced, sutured in place, and attached to a bag of normal saline with no less than 300 mmHg pressure. Laboratory blood work was drawn and sent for a Complete Blood Count and Coagulation Profile (IDEXX Preclinical Research Services, West Sacramento, Calif.). Heparin, 100 units/kg was given intravenously. An Activated Clotting Time (ACT) was drawn after 10 minutes and then every 20 minutes during the procedure with additional heparin given as needed to maintain the ACT >250 seconds to ensure adequate anticoagulation. ECG and blood pressure (Siemens Monitor, Model # 8792129E3501)) and oxygen saturation (Novametrix Tidal Wave Sp Capnography/Oximetry Model 710/715, Wallingford Conn.) were monitored during the surgery.

50 μg of NTG is administered via the guide catheter and baseline angiography performed. The Left Anterior Descending (LAD) and Left Circumflex (LCX) coronary arteries were randomized, in a blinded manner to the operator, as to which vessel receives a coated or uncoated 3.0 mm diameter stent. A 0.014 guidewire was passed into the distal coronary artery and the stent deployed at 9 atmospheres pressure via a standard balloon deployment device. Once a stent was deployed, 50 μg of NTG was given via the guiding catheter. The opposing coronary artery then had a stent placed into it. Post treatment angiograms were obtained. In 6 animals, euthanasia was accomplished after a two hour time period and the vessels perfusion fixed with formalin and sent for scanning electron microscopy to evaluate platelet adherence and acute thrombogenicity. In the remaining swine, the catheters were removed and the femoral artery and incision repaired and the animal recovered from anesthesia. Aspirin 81 mg and Plavix 75 mg were administered orally each day until the animal were sacrificed at their designated time-points. For post-operative pain management, Fentanyl patches, 75 Ug/H, were applied for 72 hours. The swine will be observed on a daily basis for signs of pain and discomfort to include but not limited to malaise, poor eating habits, lack of socialization, pain response to touch, fever, and observable infection.

At either 1,2, or 4 weeks, the subjects were sedated with Telazol®, 4-9 mg/kg, and placed under inhaled anesthesia, as stated in the above procedure. A left femoral artery cutdown was performed and the artery cannulated with a 6fr sheath. A 6fr diagnostic catheter was used to cannulate the left coronary artery, 50 μg of NTG was given via the catheter and angiograms performed. The chest opened with a sternal saw and held open with chest retractors. The heart was carefully dissected out and removed and the aortic root flushed with normal saline followed by 10% buffered formalin to perfusion fix the coronary arteries. The treated arteries were carefully dissected out and sent to CV Path, International Registry of Pathology (Gaithersburg, Md.) for histological analysis.

### Scanning Electron Microscopy Procedure

A total of 18 stents were processed for scanning electron microscopy. Scanning electron microscopy was used to evaluate the presence of thrombi, endothelial coverage, and endothelial maturity. Before processing, the stents were bisected longitudinally to expose the luminal surface and photographed. Specimens were rinsed in 0.1-mmol/L sodium cacodylate buffer (pH 7.2) and then post-fixed in 1% osmium tetroxide for 30 minutes.

Specimens were then dehydrated in a graded series of ethanol. After critical point drying, the tissue was mounted and sputter-coated with gold and specimens were visualized using a Hitachi scanning electron microscope. The percentage of endothelium was based on a visual estimate.

### Morphometry

A vessel injury score was calculated according to the Schwartz method. The cross-sectional areas (external elastic lamina [EEL], internal elastic lamina [IEL] and lumen) of each section were measured with digital morphometry. Neointimal thickness was measured as the distance from the inner surface of each stent strut to the luminal border. Percent area stenosis was calculated with the formula (Neointimal Area/IEL Area)×100). Ordinal data were collected on each stent section and included fibrin deposition, granuloma, red blood cell (RBC) and giant cell reactions around the stent struts and were expressed as a percentage of the total number of struts in each section. An overall inflammation (value 0-4) value was scored for each section. Struts with surrounding granuloma reactions were given a score of 4. Endothelial coverage was semi-quantified and expressed as the percentage of the luminal circumference covered by endothelium. The morphometric analysis for stents is reported as the mean±SD. Mean variables were compared between the groups with the use of unpaired t-tests. A value of P 20.05 was considered statistically significant.

### Radio Graphic Findings

X-rays of the vessels show good conformity of the stents in the vessel wall, including curvatures. The control stent in animal #472-B shows a focal crush artifact on the distal end of the stent.

### Histology Findings

7-Day Group; Animal # 477-A (Test): Representative sections from the proximal, mid, and distal segment of the stent show minimal neointimal incorporation over the stent surface with moderate fibrin deposition surrounding the struts. The luminal surface shows complete endothelialization with 8% (mean) cross sectional narrowing. There is focal, minimal chronic inflammation consisting of 10 or less inflammatory cells surrounding 3 to 6 struts with 35 D 47% of the struts showing giant cell reaction. There is no evidence of adventitial inflammation. Vessel wall injury was considered minimal (1), consisting of occasional IEL laceration. No malapposition of stent observed.

7-Day Group; Animal # 477-B (Control): Representative sections from the proximal, mid, and distal segment of the stent show minimal neointimal incorporation over the stent surface with moderate to marked fibrin deposition surrounding the struts. The luminal surface shows complete endothelialization with 8% (mean) cross sectional narrowing. There is focal, minimal chronic inflammation observed in the proximal segment of the stent consisting of 10 or less inflammatory cells surrounding 3 to 6 struts with 20 D 27% of the struts showing giant cell reaction. There is no evidence of adventitial inflammation. Vessel wall injury was considered minimal (1), consisting of occasional IEL laceration. No malapposition of stent observed.

7-Day Group; Animal # 478-A (Test): Representative sections from the proximal, mid, and distal segment of the stent show minimal neointimal incorporation over the stent surface with moderate fibrin deposition surrounding the struts. The luminal surface shows complete endothelialization with 8% (mean) cross sectional narrowing. There is focal, mild (2), chronic inflammation consisting of 10 or less inflammatory cells surrounding >6 struts but less than 50% of the struts with 44 D 53% of the struts showing giant cell reaction. There is no evidence of adventitial inflammation. Vessel wall injury was considered minimal (1), consisting of occasional IEL laceration. No malapposition of stent observed.

7-Day Group; Animal # 478-B (Control): Representative sections from the proximal, mid, and distal segment of the stent show minimal neointimal incorporation over the stent surface with moderate fibrin deposition surrounding the struts. The luminal surface shows complete endothelialization with 11% (mean) cross sectional narrowing. There is focal, minimal (1) to mild (2), chronic with 30 D 53% of the struts showing giant cell reaction. There is no evidence of adventitial inflammation. Vessel wall injury was considered minimal (1), consisting of occasional IEL laceration. No malapposition of stent observed.

7-Day Group; Animal # 483-A (Test): Representative sections from the proximal, mid, and distal segment of the stent show minimal neointimal incorporation over the stent surface with moderate fibrin deposition surrounding the struts. The luminal surface shows complete endothelialization with 11% (mean) cross sectional narrowing. There is focal, minimal (1) to mild (2), chronic with 21 D 35% of the struts showing giant cell reaction. There is no evidence of adventitial inflammation. Vessel wall injury was considered minimal (1) or none consisting of a single, focal IEL laceration. No malapposition of stent observed.

7-Day Group; Animal # 483-B (Control): Representative sections from the proximal, mid, and distal segment of the stent show minimal neointimal incorporation over the stent surface with moderate fibrin deposition surrounding the struts. The luminal surface shows complete endothelialization with 10% (mean) cross sectional narrowing. There is focal, minimal (1) to mild (2), chronic with 19 D 33% of the struts showing giant cell reaction. There is no evidence of adventitial inflammation. Vessel wall injury was considered minimal (1) or none consisting of a single, focal IEL laceration. No malapposition of stent observed.

7-Day Group; Animal # 495-A (Test): Representative sections from the proximal, mid, and distal segment of the stent show minimal neointimal incorporation over the stent surface with moderate fibrin deposition surrounding the struts. The luminal surface shows complete endothelialization with 9% (mean) cross sectional narrowing. There is focal minimal (1) chronic with 11 D 28% of the struts showing giant cell reaction. There is no evidence of adventitial inflammation. Vessel wall injury was considered minimal (1) or none consisting of a single, focal IEL laceration. No malapposition of stent observed.

7-Day Group; Animal # 495-B (Control): Representative sections from the proximal, mid, and distal segment of the stent show minimal neointimal incorporation over the stent surface with moderate fibrin deposition surrounding the struts. The luminal surface shows complete endothelialization with 10% (mean) cross sectional narrowing. There is focal mild (2), chronic with 17 D 33% of the struts showing giant cell reaction. There is no evidence of adventitial inflammation. Vessel wall injury was considered minimal (1) to mild (2) consisting of focal IEL and media visibly lacerated but the external elastic lamina (EEL) intact. There is no malapposition of stent observed.

7-Day Group; Animal # 496-A (Test): Representative sections from the proximal, mid, and distal segment of the stent show minimal neointimal incorporation over the stent surface with moderate fibrin deposition surrounding the struts. The luminal surface shows complete endothelialization with 12% (mean) cross sectional narrowing. There is focal mild (2), chronic with 18 D 22% of the struts showing giant cell reaction. There is minimal to mild focal, chronic inflammation in the adventitial. Vessel wall injury was considered minimal (1) consisting of focal IEL laceration. There is no malapposition of stent observed.

7-Day Group; Animal # 496-B (Control): Representative sections from the proximal, mid, and distal segment of the stent show minimal neointimal incorporation over the stent surface with moderate to marked fibrin deposition surrounding the struts. The luminal surface shows complete endothelialization with 13% (mean) cross sectional narrowing. There is focal mild (2), chronic with 16 D 30% of the struts showing giant cell reaction. There is no evidence of adventitial inflammation. Vessel wall injury was considered minimal (1) consisting of focal laceration. There is no malapposition of stent observed.

7-Day Group; Animal # 497-A (Test): Representative sections from the proximal, mid, and distal segment of the stent show minimal neointimal incorporation over the stent surface with mild to moderate fibrin deposition surrounding the struts. The luminal surface shows complete endothelialization with 12% (mean) cross sectional narrowing. There is focal mild (2) to moderate (3) chronic inflammation with 35 D 58% of the struts showing giant cell reaction. No evidence of adventitial inflammation. Vessel wall injury was considered minimal (1) consisting of focal IEL laceration. There is no malapposition of stent observed.

7-Day Group; Animal # 497-A (Control): Representative sections from the proximal, mid, and distal segment of the stent show minimal neointimal incorporation over the stent surface with mild to moderate deposition surrounding the struts. The luminal surface shows complete endothelialization with 10% (mean) cross sectional narrowing. There is focal mild (2) to moderate (3) chronic inflammation with 22 D 47% of the struts showing giant cell reaction. No evidence of adventitial inflammation. Vessel wall injury was considered minimal (1) consisting of focal IEL laceration. There is no malapposition of stent observed.

14-Day Group; Animal # 484-A (Test): Representative sections from the proximal, mid, and distal segment of the stent show mild neointimal incorporation over the stent surface with mild fibrin deposition surrounding the struts. The luminal surface shows complete endothelialization with 16% (mean) cross sectional narrowing. There is no appreciable chronic inflammation except minimal to mild giant cell reaction involving 8 D 25% of the struts. No evidence of adventitial inflammation. Vessel wall injury was considered minimal (1) consisting of focal IEL laceration. There is no malapposition of stent observed.

14-Day Group; Animal # 484-B (Control): Representative sections from the proximal, mid, and distal segment of the stent show mild neointimal incorporation over the stent surface with mild fibrin deposition surrounding the struts. The luminal surface shows complete endothelialization with 13% (mean) cross sectional narrowing. There is no appreciable chronic inflammation except minimal to mild giant cell reaction involving 16 D 25% of the struts. No evidence of adventitial inflammation. Vessel wall injury was considered minimal (1) consisting of focal IEL laceration. There is no malapposition of stent observed.

14-Day Group; Animal # 485-A (Test): Representative sections from the proximal, mid, and distal segment of the stent show mild neointimal incorporation over the stent surface with mild fibrin deposition surrounding the struts. The luminal surface shows complete endothelialization with 19% (mean) cross sectional narrowing. There is moderate (3) chronic inflammation with minimal to mild giant cell reaction involving 25 D 50% of the struts. There is minimal, focal adventitial chronic inflammation. Vessel wall injury was considered minimal (1) to mild (2) consisting of focal IEL and occasional medial laceration. There is no malapposition of stent observed.

14-Day Group; Animal # 485-B (Control): Representative sections from the proximal, mid, and distal segment of the stent show mild neointimal incorporation over the stent surface with mild fibrin deposition surrounding the struts. The luminal surface shows complete endothelialization with 22% (mean) cross sectional narrowing. There is minimal (1) chronic inflammation with minimal to mild giant cell reaction involving 16 D 30% of the struts. No evidence of adventitial inflammation. Vessel wall injury was considered minimal (1) to mild (2) consisting of focal IEL and medial laceration. There is no malapposition of stent observed.

14-Day Group; Animal # 486-A (Test): Representative sections from the proximal, mid, and distal segment of the stent show mild neointimal incorporation over the stent surface with minimal fibrin deposition surrounding the struts. The luminal surface shows complete endothelialization with 38% (mean) cross sectional narrowing. There is no appreciable chronic inflammation or giant cell reaction. Adventitial inflammation is absent. Vessel wall injury was considered minimal (1) 2) consisting of focal, occasional IEL laceration. There is no malapposition of stent observed.

14-Day Group; Animal # 486-B (Control): Representative sections from the proximal, mid, and distal segment of the stent show mild neointimal incorporation over the stent surface with mild fibrin deposition surrounding the struts. The luminal surface shows complete endothelialization with 26% (mean) cross sectional narrowing. There is minimal (1) chronic inflammation with minimal to mild giant cell reaction involving 10 D 40% of the struts. There is minimal, focal adventitial chronic inflammation. Vessel wall injury was considered minimal (1) consisting of focal IEL laceration. There is no malapposition of stent observed.

14-Day Group; Animal # 490-A (Test): Representative sections from the proximal, mid, and distal segment of the stent show mild neointimal incorporation over the stent surface with mild fibrin deposition surrounding the struts. The luminal surface shows complete endothelialization with 12% (mean) cross sectional narrowing. There is minimal (1) chronic inflammation with minimal to mild giant cell reaction involving 25 D 40% of the struts. There is minimal, focal adventitial chronic inflammation. Vessel wall injury was considered minimal (1) consisting of focal IEL and occasional medial laceration. There is no malapposition of stent observed.

14-Day Group; Animal # 490-B (Control): Representative sections from the proximal, mid, and distal segment of the stent show mild neointimal incorporation over the stent surface with mild fibrin deposition surrounding the struts. The luminal surface shows complete endothelialization with 21% (mean) cross sectional narrowing. There is mild (2) chronic inflammation with minimal to mild giant cell reaction involving 15 D 75% of the struts. No appreciable adventitial inflammation. Vessel wall injury was considered minimal (1) consisting of focal IEL and occasional medial laceration. There is no malapposition of stent observed.

14-Day Group; Animal # 493-A (Test): Representative sections from the proximal, mid, and distal segment of the stent show minimal to mild neointimal incorporation over the stent surface with mild fibrin deposition surrounding the struts. The luminal surface shows complete endothelialization with 16% (mean) cross sectional narrowing. There is moderate (3) chronic inflammation with mild to moderate giant cell reaction involving 17 D 75% of the struts. There is minimal, focal adventitial chronic inflammation. Vessel wall injury was considered minimal (1) to mild (2) consisting of focal IEL and occasional medial laceration. There is no malapposition of stent observed.

14-Day Group; Animal # 493-B (Control): Representative sections from the proximal, mid, and distal segment of the stent show minimal to mild neointimal incorporation over the stent surface with mild fibrin deposition surrounding the struts. The luminal surface shows complete endothelialization with 19% (mean) cross sectional narrowing. There is minimal (1) chronic inflammation with minimal to mild giant cell reaction involving 25 D 41% of the struts. There is minimal, focal adventitial chronic inflammation. Vessel wall injury was considered minimal (1) to mild (2) consisting of focal IEL and occasional medial laceration. There is no malapposition of stent observed.

14-Day Group; Animal # 494-A (Test): Representative sections from the proximal, mid, and distal segment of the stent show minimal to mild neointimal incorporation over the stent surface with minimal to mild fibrin deposition surrounding the struts. The luminal surface shows complete endothelialization with 14% (mean) cross sectional narrowing. There is minimal (1) chronic inflammation with minimal to mild giant cell reaction involving 5 D 2% of the struts. No evidence of adventitial chronic inflammation. Vessel wall injury was considered focal and minimal (1) consisting of occasional IEL laceration. There is no malapposition of stent observed.

14-Day Group; Animal # 494-B (Control): Representative sections from the proximal, mid, and distal segment of the stent show minimal to mild neointimal incorporation over the stent surface with mild fibrin deposition surrounding the struts. The luminal surface shows complete endothelialization with 49% (mean) cross sectional narrowing. There is marked/severe (4), granulomatous inflammation with giant cell reaction involving 100% of the struts. There is moderate adventitial chronic inflammation. Vessel wall injury was considered mild (2) consisting of focal IEL and multiple site of medial laceration. There is no malapposition of stent observed.

28-Day Group; Animal # 471-A (Test): Representative sections from the proximal, mid, and distal segment of the stent show mild to moderate neointimal incorporation over the stent surface (eccentric at the distal segment) with minimal fibrin deposition surrounding the struts (prox. and mid segment only). The luminal surface shows complete endothelialization with 40% (mean) cross sectional narrowing. There is no appreciable chronic inflammation but there is giant cell reaction involving 5 D 30% of the struts. No evidence of adventitial chronic inflammation. Vessel wall injury was considered minimal (1) to mild (2) consisting of focal IEL and EEL (occasional) laceration. There is no malapposition of stent observed.

28-Day Group; Animal # 471-B (Control): Representative sections from the proximal, mid, and distal segment of the stent show mild to moderate neointimal incorporation over the stent surface without fibrin deposition. The luminal surface shows complete endothelialization with 20% (mean) cross sectional narrowing. There is no appreciable chronic inflammation or giant cell reaction. Vessel wall injury was considered minimal (1) consisting of focal IEL laceration. There is no malapposition of stent observed.

28-Day Group; Animal # 472-A (Test) and 472-B: This is an early death animal. Representative sections from the proximal, mid, and distal segment of the stents (test and control) show a patent lumen with minimal fibrin thrombus surrounding the struts with minimal inflammatory infiltrate. Vessel wall injury was considered minimal (1), consisting of occasional IEL laceration. No malapposition of stent observed.

28-Day Group; Animal # 473-A (Test): Representative sections from the proximal, mid, and distal segment of the stent show moderate to marked neointimal incorporation over the stent surface without fibrin deposition. The luminal surface shows complete endothelialization with 54% (mean) cross sectional narrowing. There is marked (4) chronic inflammation with granulomatous and giant cell reaction involving 55 D 85% of the struts. Chronic inflammation extends to adventitial. Vessel wall injury was considered mild (2) to marked (3) consisting of large lacerations of media extending through EEL, coil wires sometimes seen in the adventitia. There is no malapposition of stent observed.

28-Day Group; Animal # 473-B (Control): Representative sections from the proximal, mid, and distal segment of the stent show mild to moderate neointimal incorporation over the stent surface with minimal to mild, focal fibrin deposition. The luminal surface shows complete endothelialization with 26% (mean) cross sectional narrowing. There is no evidence of inflammation or giant cell reaction. Vessel wall injury was minimal (1), consisting of few, focal IEL lacerations. There is no malapposition of stent observed.

28-Day Group; Animal # 474-A (Test): Representative sections from the proximal, mid, and distal segment of the stent show mild to moderate (eccentric) neointimal incorporation over the stent surface without appreciable fibrin deposition. The luminal surface shows complete endothelialization with 27% (mean) cross sectional narrowing. There is no appreciable chronic inflammation but there is minimal focal giant cell reaction involving 5 D 15% of the struts. No evidence of adventitial chronic inflammation. Vessel wall injury was considered minimal (1) consisting of focal IEL lacerations. There is no malapposition of stent observed.

28-Day Group; Animal # 474-B (Control): Representative sections from the proximal, mid, and distal segment of the stent show mild neointimal incorporation over the stent surface without appreciable fibrin deposition. The luminal surface shows complete endothelialization with 20% (mean) cross sectional narrowing. There is no appreciable chronic inflammation but there is minimal focal giant cell reaction involving 10 D 15% of the struts. No evidence of adventitial chronic inflammation. Vessel wall injury was considered minimal (1) consisting of focal, scant IEL lacerations. There is no malapposition of stent observed.

28-Day Group; Animal # 475-A (Test): Representative sections from the proximal, mid, and distal segment of the stent show mild neointimal incorporation over the stent surface without appreciable fibrin deposition. The luminal surface shows complete endothelialization with 16% (mean) cross sectional narrowing. There is no appreciable chronic inflammation but there is minimal focal giant cell reaction (only seen in the distal segment of the stent) involving 15% of the struts. No evidence of adventitial chronic inflammation. Vessel wall injury was considered minimal (1) consisting of focal, scant IEL lacerations. There is no malapposition of stent observed.

28-Day Group; Animal # 475-B (Control): Representative sections from the mid and distal segments of the stent show minimal to mild neointimal incorporation over the stent surface without appreciable fibrin deposition. The luminal surface shows complete endothelialization with 13% (mean) cross sectional narrowing. There is complete malapposition of the stent in the proximal end. There is no appreciable chronic inflammation but there is minimal focal giant cell reaction (only involving 15% of the struts. No evidence of adventitial chronic inflammation. Vessel injury was considered minimal (1) consisting of focal, IEL lacerations.

28-Day Group; Animal # 476-A (Test): Representative sections from the proximal, mid, and distal segment of the stent show mild neointimal incorporation over the stent surface without appreciable fibrin deposition. The luminal surface shows complete endothelialization with 30% (mean) cross sectional narrowing. There is no appreciable chronic inflammation but there is minimal focal giant cell reaction (only seen in the distal segment of the stent) involving 15% of the struts. No evidence of adventitial chronic inflammation. Vessel wall injury was minimal (1), consisting of focal IEL lacerations. There is no malapposition of stent observed.

28-Day Group; Animal # 476-B (Control): Representative sections from the proximal, mid, and distal segment of the stent show mild neointimal incorporation over the stent surface without appreciable fibrin deposition. The luminal surface shows complete endothelialization with 22% (mean) cross sectional narrowing. There is no appreciable chronic inflammation in the proximal and distal segments of the stent but minimal in the mid segment. Giant cell reaction was minimal, involving 7 D 25% of the struts. No evidence of adventitial chronic inflammation. Vessel wall injury was minimal (1), consisting of focal IEL lacerations. There is no malapposition of stent observed.

28-Day Group; Animal # 480-A (Test): Representative sections from the proximal, mid, and distal segment of the stent show mild neointimal incorporation over the stent surface without appreciable fibrin deposition. The luminal surface shows complete endothelialization with 20% (mean) cross sectional narrowing. There is no appreciable chronic inflammation but there is minimal focal giant cell reaction involving 6 D 20% of the struts. No evidence of adventitial chronic inflammation. Vessel wall injury was minimal (1), consisting of focal IEL lacerations. No stent malapposition observed.

28-Day Group; Animal # 481-A (Test): Representative sections from the proximal, mid, and distal segment of the stent show mild neointimal incorporation over the stent surface without appreciable fibrin deposition. The luminal surface shows complete endothelialization with 26% (mean) cross sectional narrowing. There is marked chronic inflammation with mild to marked granulomatous reaction with extension into the adventitia. Giant cell reaction is mild, and present in 20 D 47% of the stent. Vessel wall injury was minimal (1) to mild (2), consisting of focal IEL and medial lacerations. There is focal malapposition of stent in the mid segment.

28-Day Group; Animal # 481-B (Control): Representative sections from the proximal, mid, and distal segment of the stent show minimal to mild neointimal incorporation over the stent surface without appreciable fibrin deposition. The luminal surface shows complete endothelialization with 13% (mean) cross sectional narrowing. There is no appreciable chronic inflammation or giant cell reaction observed. No evidence of adventitial chronic inflammation. Vessel wall injury was minimal (1), consisting of focal IEL lacerations. No stent malapposition observed 28-Day Group; Animal # 482-A (Test): Representative sections from the proximal, mid, and distal segment of the stent show mild neointimal incorporation over the stent surface without appreciable fibrin deposition. The luminal surface shows complete endothelialization with 22% (mean) cross sectional narrowing. There is no appreciable chronic inflammation or giant cell reaction observed. No evidence of adventitial chronic inflammation. Vessel wall injury was minimal (1), consisting of focal IEL lacerations. No stent malapposition observed.

28-Day Group; Animal # 487-A (Test): Representative sections from the proximal, mid, and distal segment of the stent show mild neointimal incorporation over the stent surface without appreciable fibrin deposition. The luminal surface shows complete endothelialization with 22% (mean) cross sectional narrowing. There is no appreciable chronic inflammation but there is minimal focal giant cell reaction involving 10% of the struts (only in mid segment). No evidence of adventitial chronic inflammation. Vessel wall injury was minimal (1), consisting of focal IEL lacerations. No stent malapposition observed.

28-Day Group; Animal # 488-A (Test): Representative sections from the proximal, mid, and distal segment of the stent show mild neointimal incorporation over the stent surface with minimal, focal fibrin deposition only in the distal end of the stent. The luminal surface shows complete endothelialization with 24% (mean) cross sectional narrowing. There is no appreciable chronic inflammation or giant cell reaction observed. Vessel wall injury was minimal (1), consisting of focal IEL lacerations. No stent malapposition observed. 28-Day Group; Animal # 488-B (Control): Representative sections from the proximal, mid, and distal segment of the stent show mild neointimal incorporation over the stent surface with minimal, focal fibrin deposition. The luminal surface shows complete endothelialization with 27% (mean) cross sectional narrowing. There is no appreciable chronic inflammation but there is minimal focal giant cell reaction involving 10% of the struts (only in mid segment). No evidence of adventitial chronic inflammation. Vessel wall injury was minimal (1), consisting of focal IEL lacerations. No stent malapposition observed.

60-Day Group; Animal # 489-B (Test): Representative sections from the proximal, mid, and distal segment of the stent show minimal neointimal incorporation over the stent surface without appreciable fibrin deposition. The luminal surface shows complete endothelialization with approximately 10% (cross sectional narrowing. There is no evidence chronic inflammation or giant cell reaction. Vessel wall injury was minimal (1), consisting of focal IEL lacerations. No stent malapposition observed.

60-Day Group; Animal # 489-B (Control): Representative sections from the proximal, mid, and distal segment of the stent show moderate to marked eccentric neointimal incorporation over the stent surface without appreciable fibrin deposition. The luminal surface shows complete endothelialization with 70-80% (mean) cross sectional narrowing. There is marked (4) chronic inflammation with granulomatous and giant cell reaction involving 60% of the struts. There is marked adventitial chronic inflammation. Vessel wall injury was considered mild (2) to marked (3) consisting of large lacerations of media extending through EEL with coil wires seen in the media and close to adventitia. There is no malapposition of stent observed.

### Scanning Electron Microscopy Analysis

The first twelve (12) stents submitted for SEM (test n=6 and control n=6)) were acute explants (hours to 1-day) and consequently, separation of stent from vessel during longitudinal bisection was inevitable. Essentially, all stent struts were well expanded and apposed to the vessel walls but without any neointima formation as expected. Overall, SEM analyses of the stents surface show no apparent differences between histological changes observed in the test and the control groups. These changes consisted of focal inflammatory cell adhesions with minimal fibrin/platelet aggregations and focal areas of endothelialization. All the stents were patent.

In the 14-day (pig #501) and 28-day time points (#502 and #503), both the test and control articles showed well expanded stents with good strut apposition to the vessel wall and patent lumina without evidence of surface thrombus. Similarly, in both time points, the Tropoelastin coated stents and Bare stents showed complete coverage of luminal surface by confluent endothelial cell layer with underlying incorporation of thin neointimal growth. The endothelial cells are generally polygonal in shape with well-formed junctions. Few inflammatory cell adhesions are seen in all stents. Processing artifact changes are seen on #502 and #503 consisting of an unknown precipitate.

### CONCLUSIONS

In the 7-day group, test and control stented vessels show scant neointimal incorporation over the stent surface with mild to moderate fibrin deposition surrounding the struts. All stents show widely patent lumina with partially endothelialized luminal surface and struts well apposed to the vessel wall. In both groups, vessel wall injury was considered minimal, consisting of focal IEL lacerations, except in control stent #495-B, where there was medial lacerated. Overall, chronic inflammation was determined to be minimal to mild with the exception of stents #497-A and #497-B, which had greater than 10 inflammatory cells surrounding 50% of the struts and thus, moderate. Giant cell reaction is frequently present around the stent struts in both groups. No adventitial chronic inflammation was observed.

In the 14-day group, test and control stented vessels show minimal to mild neointimal incorporation over the stent surface with mild fibrin deposition surrounding the struts. All stents show less than 20% neointima thickness with complete endothelialization of the luminal surface and struts well apposed to the vessel wall. In both groups, vessel wall injury was considered minimal, consisting of focal IEL lacerations, except in control stent #485-A, #485-B, 486-A, #486-B, #493-A, #493-B and #494-B, where the media was focally lacerated. The degree of chronic inflammation varied amongst the two groups, from no inflammation (stent #484-A, #484-B and #486-A), to minimal inflammation (stents #485-B, #486-B, #490-A, #493-B and #490-A), to moderate (stent #485-A and 493-A) and more severe granulomatous inflammation observed in stent #494-B. Giant cell reaction was also frequently observed around the stent struts in both groups. No adventitial chronic inflammation was observed, except in stent $494-B.

In the 28-day group, test and control stented vessels show mild to moderate neointimal incorporation over the stent surface with scant fibrin deposition surrounding the struts (stent #488-A and #488-B). All stents show widely patent endothelialized luminal surface with struts well apposed to the vessel wall, except stent #475-B (proximal segment D all struts are malapposed) and stent #481-A (mid segment, two struts malapposed). In both groups, vessel wall injury was considered minimal, consisting of focal IEL lacerations, except in test stent #481-A, where the media is focally lacerated. Overall, no chronic inflammation was observed in either group, except in stent #481-A, where the mid and distal segment show marked chronic and granulomatous inflammation. Giant cell reaction was less frequent in both groups when compared with earlier time points. No adventitial chronic inflammation was observed.

Overall, morphometric analysis of Tropoelastin coated stent vs. bare stent shows significant statistical differences in neointima thickness in the 7-day time point, where mean±SD for the test article is (0.017±0.03) and control article is (0.022±0.03), resulting in a P value of 0.019. Similarly, statistical differences were present in the 7-day time point when comparing the percent of struts with fibrin between the test (85.44±8.28) and the control (97.75±4.44) groups, resulting a P value of 0.009. Furthermore, statistically differences were present when comparing percent of struts surrounded by fibrin and fibrin scores in the 14-day time point between the test vs. control, both resulting in a P value of 0.017 (Table 2). No statistically significant differences were present when comparing neointima thickness between the test and control articles in either the 14-day or 28-day time points. In addition, statistical analysis showed no significant differences when comparing the percent of endothelialization, inflammation and injury scores between test and control articles for each of the time points (7-day, 14-day and 28-day).

Human recombinant tropoelastin protein coating reduced thrombus adherence to metal stents at 7 and 14 days. Inflammation and endothelialization were not affected even though this was a human protein placed in a swine artery. Human recombinant tropoelastin proteins may be an improved and more physiologic coating with inherent favorable vascular effects and may serve as an improved platform for intravascular drug delivery over present stent and stent coating technologies.

Having described and illustrated the principles of the invention in a preferred embodiment thereof, it should be apparent that the invention can be modified in arrangement and detail without departing from such principles. I claim all modifications and variation coming within the spirit and scope of the following claims.

1. A method for producing a device implantable within a human body, comprising:
forming a biocompatible coating in situ on at least a portion of an outer surface of a substrate, wherein the biocompatible coating comprises tropoelastin.

2. The method of claim 1, wherein said biocompatible coating comprises a polymer consisting essentially of tropoelastin.

3. The method of claim 1, wherein said forming a biocompatible coating in situ on at least a portion of an outer surface of the substrate comprises cross-linking tropoelastin on the outer surface of the substrate.

4. The method of claim 3, wherein said cross-linking tropoelastin on the outer surface of the substrate comprises introducing the substrate into a cross-linking solution.

5. The method of claim 4, wherein the cross-linking solution comprises a solvent capable of substantially preventing redissolution of the tropoelastin.

6. The method of claim 5, wherein the cross-linking solution comprises a water immiscible solvent.

7. The method of claim 4, wherein the cross-linking solution comprises a suberate cross-linking agent.

8. The method of claim 1, wherein said forming a biocompatible coating in situ on at least a portion of an outer surface of the substrate comprises cross-linking tropoelastin monomers to form a polymer consisting essentially of tropoelastin.

9. The method of claim 1, wherein said forming a biocompatible coating in situ on at least a portion of an outer surface of the substrate comprises:
forming an intermediate bonding layer on at least a portion the outer surface of the substrate; and
adhering tropoelastin to an outer surface of the intermediate bonding layer.

10. The method of claim 9, wherein said adhering tropoelastin to an outer surface of the intermediate bonding layer comprises covalently bonding tropoelastin to the outer surface of the intermediate bonding layer.

11. The method of claim 9, wherein the intermediate bonding layer comprises amine groups for cross-linking tropoelastin to the outer surface of said substrate.

12. The method of claim 9, wherein the intermediate bonding layer comprises an aminosilane for cross-linking the tropoelastin monomer to the outer surface of said substrate.

13. The method of claim 1, further comprising pretreating the substrate prior to forming the biocompatible coating to form a pretreated substrate which facilitates adhering of the biocompatible coating thereto.

14. The method of claim 12, wherein said pretreating the substrate prior to forming the biocompatible coating comprises oxidizing the substrate.

15. The method of claim 13, wherein said oxidizing the substrate comprises electrochemical oxidation.

16. The method of claim 12, wherein the pretreated substrate has a contact angle which is not more than about 50% of the contact angle of the unpretreated substrate prior to pretreatment.

17. The method of claim 2, wherein the substrate coated with the tropelastin polymer has a contact angle which is at least about 150% of the contact angle of the unpretreated substrate prior to pretreatment.

18. The method of claim 1, which further includes the step of arranging the tropoelastin to form poly-tropoelastin aggregates prior to forming said biocompatible coating in situ on at least a portion of an outer surface of the substrate.

19. The method of claim 1, wherein the substrate is formed of a metallic material.

20. The method of claim 1, wherein the substrate is formed of a non-metallic material.

21. The method of claim 1, wherein the substrate is a prosthetic device.

22. The method of claim 1, wherein the substrate comprises a stent, a conduit or a scaffold.

23. The method of claim 1, wherein the biocompatible coating is formed in a substantially single layer onto the substrate.

24. The method of claim 1, wherein the biocompatible coating includes a drug for use in the human body.

25. A device implantable within a human body, comprising:
a substrate having an outer surface;
an intermediate bonding layer coating at least a portion of said outer surface of the substrate; and
an outer biocompatible layer of tropoelastin adheringly joined to the intermediate bonding layer.

26. The device of claim 25 wherein, the outer biocompatible layer of tropoelastin is cross-linked to an outer surface of the intermediate bonding layer.

27. The device of claim 25, wherein said outer biocompatible layer of tropoelastin is joined to the outer surface of the intermediate bonding layer by covalent bonding.

28. The device of claim 25, wherein said substrate comprises a pretreated substrate which facilitates adhering of the biocompatible coating thereto.

29. The device of claim 28, wherein said pretreated substrate comprises an oxidatively pretreated substrate

30. The device of claim 28, wherein said pretreated substrate is an oxidatively electrochemically pretreated substrate.

31. The device of claim 28, wherein the pretreated substrate has a contact angle which is not more than about 50% of the contact angle of an unpretreated substrate.

32. The device of claim 28, wherein the substrate adheringly coated with the tropoelastin polymer has a contact angle which is at least about 150% of the contact angle of an unpretreated substrate.

33. The device of claim 25, wherein said substrate is formed of a metallic material.

34. The device of claim 25, wherein said substrate is formed of a non-metallic material.

35. The device of claim 25, wherein said substrate is a prosthetic device.

36. The device of claim 25, wherein the intermediate bonding layer comprises cross-linkable amine groups.

37. The device of claim 25, wherein the intermediate bonding layer comprises an aminosilane.

38. The device of claim 25, wherein the tropoelastin is formed in a substantially single layer onto the bonding coating layer.

39. The device of claim 25, wherein the pretreated substrate comprises a stent, a conduit or a scaffold.

40. The device of claim 25, wherein the outer biocompatible layer of tropoelastin comprises a polymer consisting essentially of tropoelastin.

41. The device of claim 1, wherein the outer biocompatible layer of tropoelastin includes a drug for use in the human body.

42. A device implantable within a human body, comprising:
a pretreated substrate, having a pretreated outer surface capable of being adheringly coated with a layer of tropoelastin; and
an outer in-situ biocompatible layer of tropoelastin polymer adheringly joined to the pretreated substrate.
